(19) United States
(12) Patent Application Publication
(10) Pub. No.: US 2026/0042741 A1
(43) Pub. Date: Feb. 12, 2026

(54) METHODS FOR PREPARING (-)-EPICATECHIN

(71) Applicant: MARS, INCORPORATED, McLean, VA (US)

(72) Inventor: Godwin Pais, Hamden, CT (US)

(73) Assignee: MARS, INCORPORATED, McLean, VA (US)

(21) Appl. No.: 19/363,269

(22) Filed: Oct. 20, 2025

Related U.S. Application Data
(63) Continuation of application No. PCT/US2024/021155, filed on Mar. 22, 2024.
(60) Provisional application No. 63/461,058, filed on Apr. 21, 2023.

(51) Int. Cl. C07D 311/62

(52) U.S. Cl. CPC C07D 311/62

(57) ABSTRACT

Methods steps for the synthesis of (−)-epicatechin are provided herein. The synthetic methods are provided in two phases: (1) synthesis of epoxide intermediates and (2) synthesis of (−)-epicatechin.

### CROSS REFERENCE TO RELATED APPLICATIONS

This application is a continuation of International Patent Application No. PCT/US2024/021155, filed Mar. 22, 2024, which claims the benefit of priority to U.S. Provisional Application Ser. No. 63/461,058 filed Apr. 21, 2023, the contents of each of which are incorporated herein by reference in their entirety.

### FIELD

The present disclosure relates to synthesis of (−)-epicatechin.

### BACKGROUND

Catechin and epicatechin are naturally occurring polyphenolics that are widely distributed in the plant system. They are found in cocoa, tea, fruits, vegetables, and pine bark. (+)-Catechin and (−)-epicatechin are the most abundant naturally occurring epimers. For example, green tea leaves contain (−)-epicatechin and (+)-catechin. The reported biological activities of these compounds include anti-tumor activity, anti-mutagenic activity, and antioxidant activity. However, isolation of stereospecific catechin derivatives, such as e.g., (−)-epicatechin, often requires difficult purification steps to separate the compound of interest from other epimers and/or structurally similar compounds present in the natural extracts. As such, there is a need for efficient synthetic methods for the large-scale production of catechin and epicatechin monomers from commercially available materials at the purity levels required for scale-up syntheses.

### SUMMARY

The purpose and advantages of the disclosed subject matter will be set forth in and are apparent from the description that follows, as well as will be learned by practice of the disclosed subject matter. Additional advantages of the disclosed subject matter will be realized and attained by the devices particularly pointed out in the written description and claims hereof.

To achieve these and other advantages and in accordance with the purpose of the disclosed subject matter, as embodied and broadly described, the disclosed subject matter includes various methods for preparing (−)-epicatechin.

In certain embodiments, the presently disclosed subject matter provides a method for preparing (−)-epicatechin. In certain embodiments the method includes providing a compound of Formula V, wherein X is O or S, R₃ is Ph, Bn, or CH(CH₃)₂, and R₄ is Ph or H:

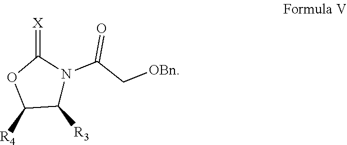

In certain embodiments, the method includes further providing a compound of Formula II wherein R₁ is MOM or Bn:

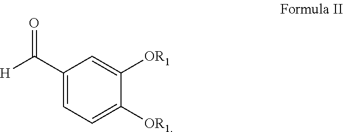

In certain embodiments, the method includes coupling the compounds of Formula II and V to form a first intermediate, wherein the first intermediate comprises a secondary alcohol. In certain embodiments, the method includes protecting the secondary alcohol in the first intermediate to form a second intermediate. In certain embodiments, the method includes reducing the second intermediate to form a third intermediate, wherein the third intermediate comprises a primary alcohol. In certain embodiments, the method includes removing the Bn group of Formula V to form a fourth intermediate. In certain embodiments, the method includes selectively forming a sulfonate ester from the primary alcohol of the fourth intermediate to form a fifth intermediate. In certain embodiments, the method includes epoxidizing the fifth intermediate to form a sixth intermediate. In certain embodiments, the method includes alkylating the sixth intermediate to form a seventh intermediate, wherein the seventh intermediate comprises a primary alcohol. In certain embodiments, the method includes protecting the primary alcohol of the seventh intermediate to form an eighth intermediate. In certain embodiments, the method includes cyclizing the eighth intermediate to form a ninth intermediate. In certain embodiments, the method includes deprotecting the ninth intermediate to form a tenth intermediate. In certain embodiments, the method includes deprotecting the tenth intermediate to form (−)-epicatechin.

In certain embodiments, X is S and R₃ is Ph. In particular embodiments, R₁ is MOM.

In certain embodiments, the alcohol of the first intermediate is protected with a silyl group. In particular embodiment, the silyl group is t-butyldimethylsilyl.

In certain embodiments, the sulfonate ester of is —OTs, —OMes, or —OTf. In particular embodiments, the sulfonate ester is —OTs.

In certain embodiments, the sixth intermediate is alkylated with a compound of Formula VII wherein X is F, Br, I, Cl, N, S, or B and R₃ is a Bn or MOM protecting group, to form the seventh intermediate:

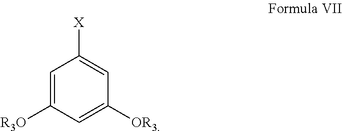

In particular embodiments, X is F and R₃ is Bn.

In certain embodiments, the seventh intermediate is protected with a MOM or Bn protecting group. In particular embodiments, the seventh intermediate is protected with a MOM protecting group.

In certain embodiments, the deprotection of the ninth intermediate is a debenzylation. In particular embodiments, the sixth intermediate is the syn-epoxide intermediate.

In certain embodiments, the presently disclosed subject matter provides a method for preparing (−)-epicatechin. In certain embodiments, the method includes providing (S)-2-(Benzyloxy)-1-(4-phenyl-2-thioxooxazolidin-3-yl)ethan-1-one and 3,4-bis(methoxymethoxy)-benzaldehyde. In certain embodiments, the method includes adding chloromethyl methyl ether to produce (2S,3R)-2-(benzyloxy)-3-(3,4-bis(methoxymethoxy)phenyl)-3-hydroxy-1-((S)-4-phenyl-2-thioxooxazolidin-3-yl)propan-1-one. In certain embodiments, the method includes protecting (2S,3R)-2-(benzyloxy)-3-(3,4-bis(methoxymethoxy)phenyl)-3-hydroxy-1-((S)-4-phenyl-2-thioxooxazolidin-3-yl)propan-1-one with TBSCl to produce (2S,3R)-2-(benzyloxy)-3-(3,4-bis(methoxymethoxy)phenyl)-3-((tert-butyldimethylsilyl)oxy)-1-((S)-4-phenyl-2-thioxooxazolidin-3-yl)propan-1-one. In certain embodiments, the method includes reducing (2S,3R)-2-(benzyloxy)-3-(3,4-bis(methoxymethoxy)phenyl)-3-((tert-butyldimethylsilyl)oxy)-1-((S)-4-phenyl-2-thioxooxazolidin-3-yl)propan-1-one with LiBH₄ to produce (2R,3R)-2-(benzyloxy)-3-(3,4-bis(methoxymethoxy)phenyl)-3-((tert-butyldimethylsilyl)oxy)propan-1-ol. In certain embodiments, the method includes deprotecting (2R,3R)-2-(benzyloxy)-3-(3,4-bis(methoxymethoxy)phenyl)-3-((tert-butyldimethylsilyl)oxy)propan-1-ol with Pd/C under H₂ to produce (2R,3R)-3-(3,4-bis(methoxymethoxy)phenyl)-3-((tert-butyldimethylsilyl)oxy)propane-1,2-diol. In certain embodiments, the method includes tosylating (2R,3R)-3-(3,4-Bis(methoxymethoxy)phenyl)-3-((tert-butyldimethylsilyl)oxy)propane-1,2-diol with tosylic acid to produce (2R,3R)-3-(3,4-bis(methoxymethoxy)phenyl)-3-((tert-butyldimethylsilyl)oxy)-2-hydroxypropyl 4-methylbenzenesulfonate. In certain embodiments, the method includes epoxidizing (2R,3R)-3-(3,4-bis(methoxymethoxy)phenyl)-3-((tert-butyldimethylsilyl)oxy)-2-hydroxypropyl 4-methylbenzenesulfonate using potassium carbonate in methanol to produce ((R)-(3,4-bis(methoxymethoxy)phenyl)((R)-oxiran-2-yl)methoxy)(tert-butyl)dimethylsilane. In certain embodiments, the method includes alkylating ((R)-(3,4-bis(methoxymethoxy)phenyl)((R)-oxiran-2-yl)methoxy)(tert-butyl)dimethylsilane with (((5-fluoro-1,3-phenylene)bis(oxy))bis(methylene))dibenzene using n-butyl lithium to produce (1R,2R)-3-(2,4-bis(benzyloxy)-6-fluorophenyl)-1-(3,4-bis(methoxymethoxy)phenyl)-1-((tert-butyldimethylsilyl)oxy)propan-2-ol. In certain embodiments, the method includes, protecting (1R,2R)-3-(2,4-bis(benzyloxy)-6-fluorophenyl)-1-(3,4-bis(methoxymethoxy)phenyl)-1-((tert-butyldimethylsilyl)oxy)propan-2-ol with MOMCl to produce (5R,6R)-5-{[2,4-bis(benzyloxy)-6-fluorophenyl]methyl}-6-[3,4-bis(methoxymethoxy)phenyl]-8,8,9,9-tetramethyl-2,4,7-trioxa-8-siladecane. In certain embodiments, the method includes deprotecting (5R,6R)-5-{[2,4-bis(benzyloxy)-6-fluorophenyl]methyl}-6-[3,4-bis(methoxymethoxy)phenyl]-8,8,9,9-tetramethyl-2,4,7-trioxa-8-siladecane with TBAF to product (1R,2R)-3-(2,4-bis(benzyloxy)-6-fluorophenyl)-1-(3,4-bis(methoxymethoxy)phenyl)-2-(methoxymethoxy)propan-1-ol. In certain embodiments, the method includes cyclizing (1R,2R)-3-(2,4-bis(benzyloxy)-6-fluorophenyl)-1-(3,4-bis(methoxymethoxy)phenyl)-2-(methoxymethoxy)propan-1-ol using potassium hydride to product (2R,3R)-5,7-bis(benzyloxy)-2-(3,4-bis(methoxymethoxy)phenyl)-3-(methoxymethoxy)chromane. In certain embodiments, the method includes deprotecting (2R,3R)-5,7-bis(benzyloxy)-2-(3,4-bis(methoxymethoxy)phenyl)-3-(methoxymethoxy)chromane with HCl to produce 4-((2R,3R)-5,7-bis(benzyloxy)-3-hydroxychroman-2-yl)benzene-1,2-diol. In certain embodiments, the method includes deprotecting 4-((2R,3R)-5,7-bis(benzyloxy)-3-hydroxychroman-2-yl)benzene-1,2-diol with Pd/C under H₂ to give (−)-epicatechin.

### BRIEF DESCRIPTION OF THE DRAWINGS

FIG. 1 shows general steps associated with the synthesis of the (−)epicatechin derivatives as disclosed herein.

FIG. 2 shows general steps associated with the synthesis of the (−)epicatechin derivatives as disclosed herein.

FIG. 3 shows general steps associated with the synthesis of the (−)epicatechin derivatives as disclosed herein.

FIG. 4 shows general steps associated with the synthesis of the (−)epicatechin derivatives as disclosed herein.

FIG. 5 shows general steps associated with the synthesis of the (−)epicatechin derivatives as disclosed herein.

FIG. 6 shows general steps associated with the synthesis of the (−)epicatechin derivatives as disclosed herein.

FIG. 7 shows general steps associated with the synthesis of the (−)epicatechin derivatives as disclosed herein.

### DETAILED DESCRIPTION

Stereospecific catechin derivatives, including (−)-epicatechin and (+)-epicatechin, have reported biological activities that include anti-tumor activity, anti-mutagenic activity, antioxidant activity, and others. However, obtaining each of these compounds at an industrial scale and purity required for health applications such as, e.g., pharmaceutical applications remains difficult. The present disclosure provides a synthesis of stereo-pure epimers of catechin from commercially available starting materials to address the preceding needs.

For clarity, but not by way of limitation, the detailed description of the presently disclosed subject matter is divided into the following subsections:
I. Definitions;
II. Catechin Epimers;
III. Synthetic Processes; and
IV. Consumer Products.

### I. Definitions

The terms used in this specification generally have their ordinary meanings in the art, within the context of this disclosure and in the specific context where each term is used. Certain terms are discussed below, or elsewhere in the specification, to provide additional guidance to the practitioner in describing the compositions and methods of the present disclosure and how to make and use them.

For purposes of interpreting this specification, the following definitions will apply and whenever appropriate, terms used in the singular will also include the plural and vice versa. In the event that any definition set forth below conflicts with any document incorporated herein by reference, the definition set forth below shall control.

As used herein, the use of the word “a” or “an” when used in conjunction with the term “comprising” in the claims and/or the specification can mean “one,” but it is also consistent with the meaning of “one or more,” “at least one,” and “one or more than one.”

The terms “comprise(s),” “include(s),” “having,” “has,” “can,” “contain(s),” and variants thereof, as used herein, are intended to be open-ended transitional phrases, terms or words that do not preclude additional acts or structures. The present disclosure also contemplates other embodiments “comprising,” “consisting of” and “consisting essentially of,” the embodiments or elements presented herein, whether explicitly set forth or not.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value.

The term “alkyl” refers to a straight or branched C₁-C₂₀ hydrocarbon group consisting solely of carbon and hydrogen atoms, containing no unsaturation, and which is attached to the rest of the molecule by a single bond, e.g., methyl, ethyl, n-propyl, 1-methylethyl (isopropyl), n-butyl, n-pentyl, 1,1-dimethylethyl (t-butyl).

The term “alkenyl” refers to a C₂-C₂₀ aliphatic hydrocarbon group containing at least one carbon-carbon double bond and which may be a straight or branched chain, e.g., ethenyl.

The term “cycloalkyl” denotes an unsaturated, non-aromatic mono- or multicyclic hydrocarbon ring system (containing, for example, C₃-C₆) such as cyclopropyl, cyclobutyl, cyclopentyl, cyclohexyl.

The term “aryl” refers to aromatic radicals having in the range of about 6 to about 14 carbon atoms such as phenyl or biphenyl.

The term “aryl alkyl” refers to an aryl group as defined above directly bonded to an alkyl group as defined above, e.g., —CH₂C₆H₅, and —C₂H₄C₆H₅. As used herein the term “benzyl” or “Bn” commonly refers to a phenyl group (“Ph”) with a single alkyl group. As used herein Bn is —CH₂C₆H₆ or —CH₂Ph.

As used herein the term “chiral auxiliary” refers to a chemical compound used to control the stereochemical outcome of a reaction by integrating the chiral auxiliary into a compound then removing the chiral auxiliary after the desired stereochemistry is achieved during a synthesis.

As used herein, the term “cyclization” refers to a chemical reaction by which the atoms of one or more compounds become a closed ring.

The term “heterocyclic” refers to a stable 3- to 15-membered ring radical, which consists of carbon atoms and one or more, for example, from one to five, heteroatoms selected from the group consisting of nitrogen, oxygen and sulfur. For purposes of this application, the heterocyclic ring radical may be a monocyclic or bicyclic ring system, which may include fused or bridged ring systems, and the nitrogen, carbon, oxygen or sulfur atoms in the heterocyclic ring radical may be optionally oxidized to various oxidation states.

The term “heteroaryl” refers to a heterocyclic ring wherein the ring is aromatic.

As used herein the term “ether” commonly refers to two alkyl groups bonded to an oxygen atom.

As used herein, the term “intermediate” refers to a compound that is formed by a reaction in multi-step synthesis and is then used in a subsequent step of the said multi-step synthesis. In certain embodiments, the intermediate can be isolated and purified using methods known to a person of ordinary skill in the art before using in a subsequent step.

In certain other embodiments, the intermediate can be used in the subsequent step without further purification. In certain embodiments, the intermediate can be prepared in situ and then used for a subsequent step.

As used herein, the term “isomers” refers to different compounds that have the same molecular formula but differ in arrangement and configuration of the atoms. Also, as used herein, the term “stereoisomer” refers to any of the various stereo isomeric configurations which can exist for a given compound of the presently disclosed subject matter and includes geometric isomers. It is understood the compound of the present disclosure contains double bonds, where the substituents can be E or Z configuration. Furthermore, as used herein, the term “epimer” refers to either of two stereoisomers that differ in the arrangement of groups on a single asymmetric carbon atom (such as the first chiral center of a sugar's carbon chain). For instance, but not by the way of limitation, (−)-epicatechin and (+)-epicatechin are epimers. Also, as used herein, the terms “constitutional isomers” refers to different compounds which have the same numbers of, and types of, atoms but the atoms are connected differently. The term “optical isomers”, as used herein, refers two compounds which contain the same number and kinds of atoms, and bonds (i.e., the connectivity between atoms is the same), and different spatial arrangements of the atoms, but which have non-superimposable mirror images. Each non-superimposable mirror image structure is called an enantiomer.

As used herein, the term “leaving group” refers to an atom or a molecular fragment that departs with a pair of electrons in heterolytic bond cleavage. Leaving groups can be anions, cations, or neutral molecules. As known to a person of ordinary skill in the art, the more stable an atom or group of atoms can be on its own, the better of a leaving group it is. For instance, a good leaving group can be a conjugate base of a strong acid. A leaving group is a neutral molecule such as e.g., water or ammonia in certain embodiments.

As used herein the term “organolithium” refers to a compound where there is at least one carbon bonded to at least one lithium.

As used herein, the term “oxidation” or “oxidized” refers to a chemical reaction in which a chemical species loses electrons.

As used herein, the term “protecting group” refers to a reversibly formed derivative of an existing functional group in a molecule. The protective group is temporarily attached to decrease reactivity so that the protected functional group does not react under synthetic conditions to which the molecule is subjected in one or more subsequent steps. The protecting group can be removed through a deprotection step once the selective synthetic steps are completed.

As used herein, the terms “to protect” and “protecting” refer to a synthesis step where a functional group is reversibly and temporarily derivatized to decrease the reactivity of said functional group. For instance, but not by way of limitation, “protecting a hydroxy group” refers to performing a synthesis step where a reagent, such as e.g., tert-butyldimethylsilyl chloride or chloromethyl methyl ether, is added to a compound having a hydroxy group to convert the hydroxy group to a tert-butyldimethylsilyl or a methoxymethyl ether. As would be known to a person of ordinary skill in the art, other reagents that can install the same or different protecting groups can also be used to protect a functional group.

As used herein, the term “reduction” or “reduced” refers to a chemical reaction in which a chemical species gains electrons.

As used herein the term “sulfonate” commonly refers to a sulfur atom that is double-bonded to two oxygen atoms, single-bonded to one oxygen atom, and single-bonded to a further group. A “sulfonylation reaction” refers to the addition of a sulfonate group. Common sulfonate compounds include mesylate (“Mes”), tosylate (“Ts”), or triflate (“Tf”). As used herein, “mesyl” refers to methanesulfonyl, “trifyl” refers to trifluoromethanesulfonyl, and “tosyl” refers top-toluenesulfonyl. As used herein, “tosylate” or “tosyl group” refers top-toluenesulfonate and “tosylation” refers to the addition of a tosyl group. In certain embodiments, the tosyl group is added with tosyl chloride (TsCl). As used herein, “sulfonate ester” commonly refers to a sulfonate where the oxygen that is single-bonded to the sulfur atom is bonded to a further group. Common sulfonate ester compounds include “OMes”, “OTs”, or “OTf”.

### II. Catechin Epimers

Catechin is a natural phenol present in various sources, including many herbs, fruits, vegetables, beverages, algae, and confectionary items. Cocoa, tea, and certain pome fruits are the main sources of catechins in the diet.

Catechin is a flavan-3-ol, which belongs to the chemical family of flavonoids. Catechin has two benzene rings (called the A- and B-rings) and a dihydropyran heterocycle (the C-ring) with a hydroxyl group on carbon 3, as shown by Formula I:

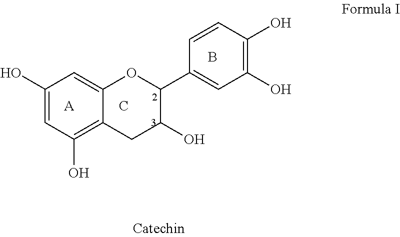

There are two chiral centers on the molecule on carbons 2 and 3, and as such the molecule has four diastereomers, referred herein as epimers. These four epimers are (+)-catechin, (−)-epicatechin, (−)-catechin, and (+)-epicatechin shown below as Formulae IA-ID:

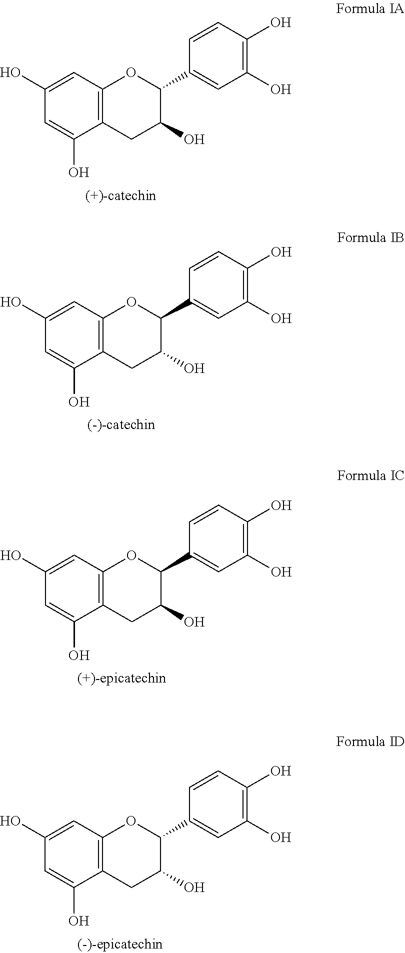 [SMILES: Oc1cc(O)c2c(c1)O[C@H](c1ccc(O)c(O)c1)[C@@H](O)C2 ; Oc1cc(O)c2c(c1)O[C@@H](c1ccc(O)c(O)c1)[C@H](O)C2 ; Oc1cc(O)c2c(c1)O[C@@H](c1ccc(O)c(O)c1)[C@@H](O)C2 ; Oc1cc(O)c2c(c1)O[C@H](c1ccc(O)c(O)c1)[C@H](O)C2]

Although the four epimers of catechin are similar in structure, they have been shown to have certain markedly different activities. For instance, Tsuchiya has reported that the epimers show various biological activities through their interactions with cellular membranes. For instance, both (−)-epicatechin and (+)-epicatechin were shown to be more effective for reducing membrane fluidity than (+)-catechin and (−)-catechin. Reversed-phase chromatographic evaluation showed that (−)-epicatechin and (+)-epicatechin were more hydrophobic than (−)-catechin and (+)-catechin, although hydrophobicity was not distinguishable between optical isomers. See, Tsuchiya “Stereospecificity in membrane effects of catechins” Chem. Biol. Interact 2001, 134, 41-54, which is incorporated herein by reference.

The isolation of the stereospecific catechin epimers is often very difficult. Thus, the synthesis of the (−)-epicatechin provided in the present disclosure allows the formation of a single epimer for application to various consumer products.

### III. Synthetic Processes

The present disclosure provides synthesis methods (−)-epicatechin. In certain embodiments, the synthesis of (−)-epicatechin can include two phases—a first phase preparing the epoxide intermediates; and a second phase converting the epoxide intermediate to the desired (−)-epicatechin. Each of the phases are further described below.

### Phase 1: Preparation of Epoxide Intermediates

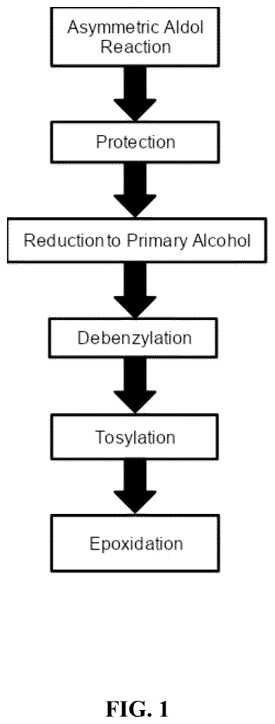

In certain embodiments, the first phase includes preparation of the syn-epoxide intermediate. In certain embodiments, the first phase includes preparation of the anti-epoxide intermediate. In certain embodiments, the synthesis of the epoxide intermediate proceeds following the general steps as outlined in FIGS. 1-5 and further discussed below.

In certain embodiments, the commercially available starting material is 3,4-dihydroxybenzaldehyde. In particular embodiments, the hydroxy groups of the compound of 3,4-dihydroxybenzaldehyde are first protected using one or more protecting groups commonly known in the literature, including methoxymethyl ether (OMOM), tetrahydropyranyl ether, t-butyl ether, allyl ether, benzyl ether (OBn), triisopropylsilyl ether (OTIPS) or t-butyldimethylsilyl ether (OTBS). In certain particular embodiments, the protecting group is benzyl ether. In certain particular embodiments, the protecting group is methoxymethyl ether. In particular embodiments, R₁ is methoxymethyl (MOM) or benzyl (Bn). In certain embodiments, chloromethyl methyl ether is used to protect hydroxy groups of the compound of Formula II.

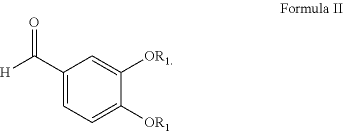

### Phase 1.1: Synthesis of Syn-E Poxide Intermediate

In certain embodiments, the syn-epoxide intermediate is formed. The syn-epoxide intermediate is a compound of Formula III where R₁ and R₂ are H or a protecting group. In particular embodiments, R₁ and R₂ are the same. In particular embodiments, R₁ and R₂ are different. In certain embodiments, R₁ and R₂ can be a H, methoxymethyl, tetrahydropyranyl, t-butyl, allyl, benzyl, triisopropylsilyl, or t-butyldimethylsilyl group. In particular embodiments, R₂ is H or t-butyldimethylsilyl and R₁ is methoxymethyl or benzyl.

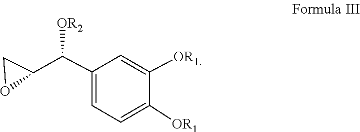

### Phase 1.1.1: 7 Step Synthesis of Syn-Epoxide Intermediate

In certain embodiments, the general synthesis of syn-epoxide intermediates is outlined in FIG. 1. As shown in FIG. 1, the synthesis includes 7 steps. In particular, the synthesis includes a coupling reaction to form a chiral auxiliary compound. The chiral auxiliary compound further undergoes an asymmetric aldol reaction. Subsequently, to the reaction product a protecting group is added and then the chiral auxiliary is removed from the compound by a reduction reaction. The resulting product then undergoes a debenzylation reaction followed by a sulfonation reaction. Finally, the desired syn-epoxide intermediate is formed.

In certain embodiments, the second starting material for synthesis of (−)-epicatechin is a compound of Formula III where X is S or O, R₃ is Ph, Bn, or CH(CH₃)₂, and R₄ is Ph or H.

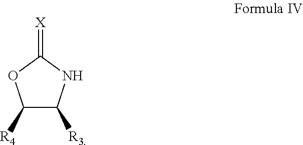

In certain embodiments, the compound of Formula IV is converted to a chiral auxiliary compound. In certain embodiments, the compound of Formula I is coupled with benzyloxyacetic acid or benzyloxyacetyl chloride using a coupling agent. Particularly, the compound of Formula I is coupled with benzyloxyacetic acid using a coupling agent. In certain embodiments, the coupling agent is 3-{[(Ethylimino)methylidene]amino}-N,N-dimethylpropan-1-amine (EDCI), (2-(1H-benzotriazol-1-yl)-1,1,3,3-tetramethyluronium hexafluorophosphate (HBTU), benzotriazol-1-yloxytris(dimethylamino)phosphonium hexafluorophosphate (BOP), benzotriazol-1-yloxytripyrrolidinophosphonium hexafluorophosphate (PyBOP), propylphosphonic anhydride (T3P), or N,N′-Dicyclohexylcarbodiimide (DCC). In particular embodiments, the coupling agent is EDCI. The coupling reaction is illustrated in Scheme I.

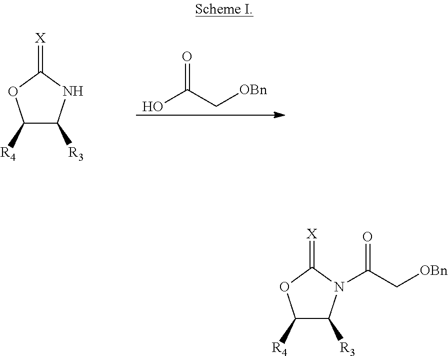

In certain embodiments, the chiral auxiliary is the compound of Formula V wherein X is O or S, R₃ is Ph, Bn, or CH(CH₃)₂, and R₄ is Ph or H.

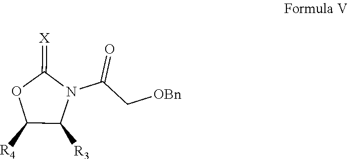

In certain embodiments, the protected 3,4-dihydroxybenzaldehyde and the chiral auxiliary are coupled in an asymmetric aldol reaction. In certain embodiments, a titanium enolate is formed from the chiral auxiliary. In particular embodiments, the titanium enolate is formed by submitted the chiral auxiliary to titanium tetrachloride. In certain embodiments, the titanium enolate is subjected to a base and the protected 3,4-dihydroxybenzaldehyde to form the anti-aldol product. In particular embodiments, the base is triethylamine. The asymmetric aldol reaction is illustrated in Scheme II.

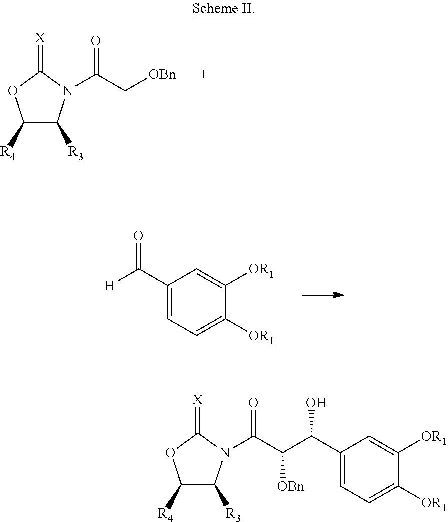

In certain embodiments, the resulting compound is further protected using one or more silyl protecting groups commonly known in the literature, including triispropylsilyl ether, t-butyldiphenylsilyl ether, or t-butyldimethylsilyl ether. In particular embodiments, the protecting group is t-butyldimethylsilyl ether. In certain embodiments, t-butyldimethylsilyl chloride is used to protect the hydroxy groups of the resulting compound. The protection reaction is illustrated in Scheme III.

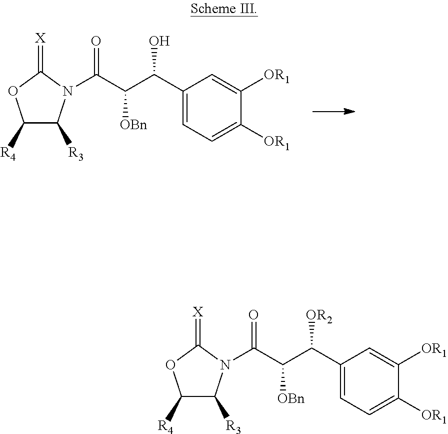

In certain embodiments, the resulting compound is subjected to a reduction reaction to remove the chiral auxiliary. In certain embodiments, the resulting compound is subjected to a reducing agent to form the resulting primary alcohol. Various reducing agents can be used to conduct this reduction such as but not limited to lithium borohydride. The reduction reaction is illustrated in Scheme IV.

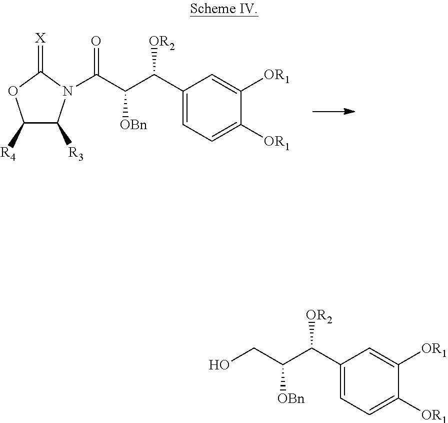

In certain embodiments, the resulting compound further undergoes debenzylation. In certain embodiments, a palladium catalyst in the presence of hydrogen gas is used for debenzylation. In certain embodiments, the palladium catalyst can be palladium on carbon (Pd/C) or palladium hydroxide on carbon (Pd(OH)₂/C). In particular embodiments, the palladium catalyst is Pd(OH)₂/C in the presence of hydrogen gas. The debenzylation reaction is illustrated in Scheme V.

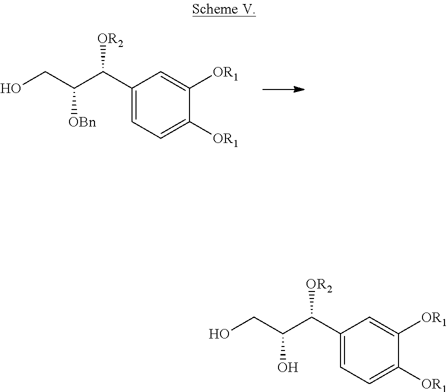

In certain embodiments, the primary alcohol of the above compound is then converted to a leaving group. In certain embodiments, the leaving group is mesylate, tosylate, triflate, bromide or chloride. In certain embodiments, the compound can be treated with a mesyl chloride, tosyl chloride, or trifyl chloride in the presence of at least one catalyst and a base. In particular embodiments, the compound is treated with tosyl chloride in the presence of the catalysts dibutyltin oxide and 4-dimethylaminopyridine (DMAP) and the base triethylamine to afford the sulfonate ester. The sulfonation reaction is illustrated in Scheme VI.

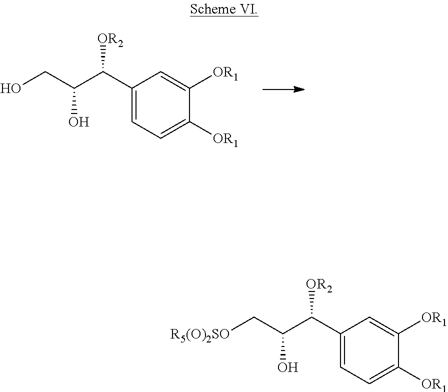

In certain embodiments, the resulting compound undergoes a further epoxidation reaction. In certain embodiments, the compound is treated with a base commonly known in the literature including potassium carbonate, potassium hydroxide, sodium hydroxide, sodium methoxide, lithium bis(trimethylsilyl)amide, ammonium hydroxide, sodium carbonate, cesium carbonate, potassium tert-butoxide. In particular embodiments, the compound is treated with potassium carbonate to afford the syn-epoxide intermediate. The epoxidation is illustrated in Scheme VII.

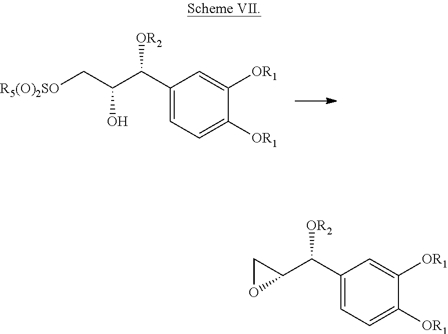

### Phase 1.1.2: 2 Step Synthesis of Syn-Epoxide

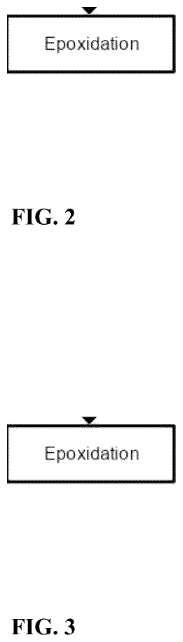

In certain embodiments, the general synthesis of syn-epoxide intermediates is outlined in FIG. 2. As shown in FIG. 2, the synthesis includes 2 steps. In particular, the synthesis includes an asymmetric addition followed by an epoxidation resulting in the formation of the desired syn-epoxide intermediate.

In certain embodiments, the protected 3,4-dihydroxybenzaldehyde can undergo an asymmetric addition with an alkene. In certain embodiments, the asymmetric addition can be carried out with vinyl trimethoxysilane in the presence of a binaphthyl catalyst. In particular embodiments, the binaphthyl catalyst is (R)-DTBM-SEGPHOS. In certain embodiments, the asymmetric addition can be further completed in the presence of a copper catalyst. The asymmetric addition is illustrated in Scheme VIII.

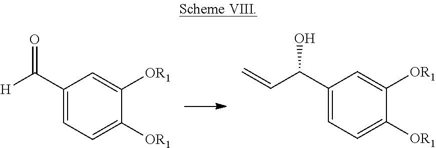

In certain embodiments, the resulting compound undergoes a further epoxidation reaction. In certain embodiments, the compound is treated with a titanium catalyst and an oxidant. In particular embodiments, the catalyst is titanium-salalen and the peroxide is hydrogen peroxide to afford the syn-epoxide intermediate. The epoxidation is illustrated in Scheme IX.

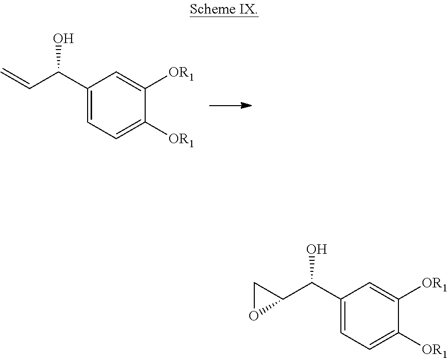

### Phase 1.2: Synthesis of Anti-Epoxide Intermediate

In certain embodiments, the anti-epoxide intermediate is formed. The anti-epoxide intermediate is a compound of Formula VI where R₁ and R₂ are H or a protecting group. In particular embodiments, R₁ and R₂ are the same. In particular embodiments, R₁ and R₂ are different. In certain embodiments, R₁ and R₂ can be H, methoxymethyl, tetrahydropyranyl, t-butyl, allyl, benzyl, triisopropylsilyl, or t-butyldimethylsilyl. In particular embodiments, R₂ is H or t-butyldimethylsilyl and R₁ is methoxymethyl or benzyl.

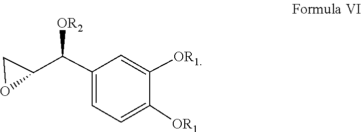

### Phase 1.2.1: 2 Step Synthesis of Anti-Epoxide

In certain embodiments, the general synthesis of anti-epoxide intermediates is outlined in FIG. 3. As shown in FIG. 3, the synthesis includes 2 steps. In particular, the synthesis includes a Grignard reaction followed by an epoxidation resulting in the formation of the desired anti-epoxide intermediate.

In certain embodiments, the protected 3,4-dihydroxybenzaldehyde can undergo a Grignard reaction. In certain embodiments, the Grignard reaction can be carried out with vinylmagnesium bromide to afford the desired product. The Grignard reaction is illustrated in Scheme X.

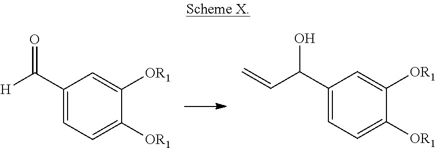

In certain embodiments, the resulting compound undergoes a Sharpless Epoxidation reaction. In certain embodiments, the compound is treated with a chiral catalyst and an oxidant. In particular embodiments, the chiral catalyst is formed from titanium tetraisopropoxide and (+)-diisopropyl tartrate ((+)-DIPT). In particular embodiments, the compound is further treated with the oxidant tert-butyl hydroperoxide (TBHP) to afford the anti-epoxide intermediate. The epoxidation is illustrated in Scheme XI.

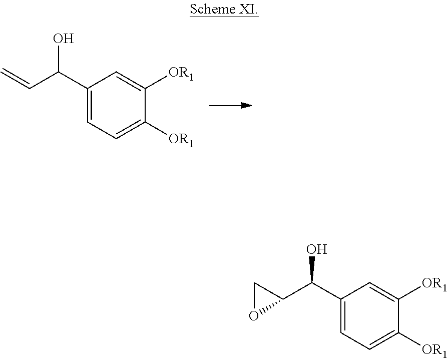

Phase 1.2.2: 5 Step Synthesis of Anti-Epoxide from 3,4-dihydroxybenzaldehyde

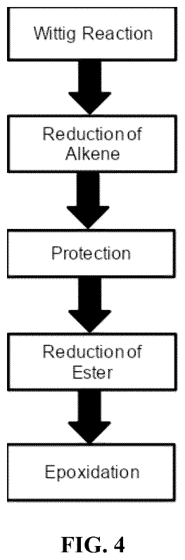

In certain embodiments, the general synthesis of anti-epoxide intermediates is outlined in FIG. 4. As shown in FIG. 4, the synthesis includes 5 steps. In particular, the synthesis includes a Wittig reaction followed by the reduction of an alkene. Subsequently, the product can undergo a protection reaction then the reduction of the ester. Finally, this is followed by an epoxidation resulting in the formation of the desired anti-epoxide intermediate.

In certain embodiments, the protected 3,4-dihydroxybenzaldehyde can undergo a Wittig reaction. In certain embodiments, the Wittig reaction is carried out using ethoxycarbonylmethyl(triphenyl)phosphonium bromide to afford the desired product. The Wittig reaction is illustrated in Scheme XII.

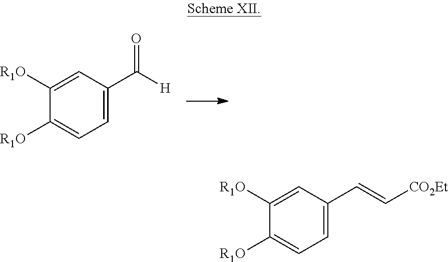

In certain embodiments, the resulting product can undergo alkene reduction. In certain embodiments, the alkene is reduced using an organolithium reagent and an acid. In particular embodiments, the alkene is reduced with the organolithium reagent lithium bromide and the acid sulfuric acid to afford the desired product. The reduction reaction is illustrated in Scheme XIII.

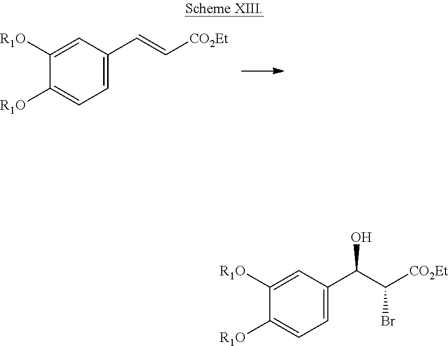

In certain embodiments, the resulting compound is further protected using one or more silyl protecting groups commonly known in the literature, including triispropylsilyl ether, t-butyldiphenylsilyl ether or t-butyldimethylsilyl ether. In certain particular embodiments, the protecting group is t-butyldimethylsilyl ether. In certain embodiments, t-butyldimethylsilyl chloride is used to protect the hydroxy groups of the resulting compound. The protection reaction is illustrated in Scheme XIV.

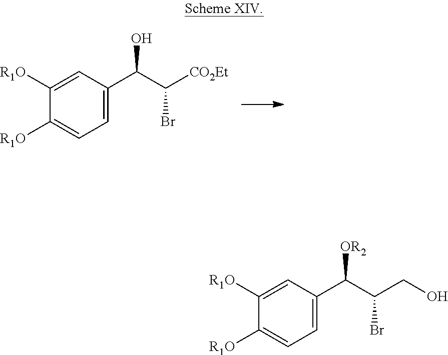

In certain embodiments, the resulting product can undergo reduction reaction. In certain embodiments, the ester is reduced using a borohydride reagent. In certain embodiments, the borohydride reagent can be sodium borohydride or lithium borohydride. In particular embodiments, the ester can be reduced with the organolithium reagent lithium bromide and the acid sulfuric acid to afford the desired product. The reduction reaction is illustrated in Scheme XV.

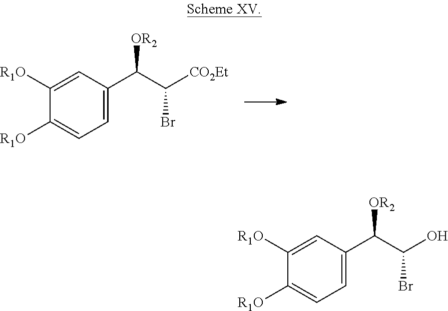

In certain embodiments, the resulting compound undergoes an epoxidation reaction. In certain embodiments, the compound is treated with a base. In certain embodiments, the base is sodium hydroxide, sodium hydride, lithium hydroxide, potassium tert-butoxide, sodium carbonate, or potassium hydroxide. In particular embodiments, the compound is treated with sodium hydroxide to afford the desired anti-epoxide. The epoxidation is illustrated in Scheme XVI.

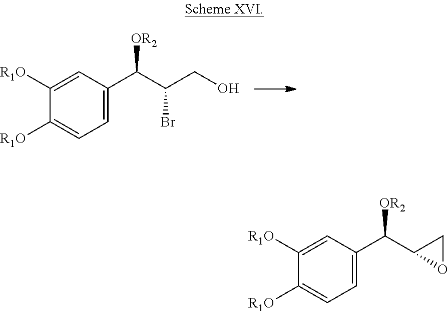

Phase 1.2.3: 5 Step Synthesis of Anti-Epoxide from Tartaric Acid

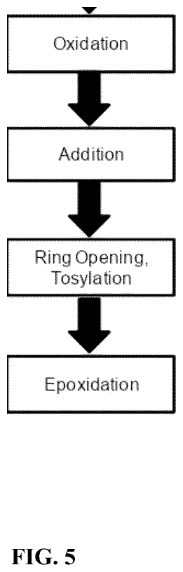

In certain embodiments, the general synthesis of anti-epoxide intermediates is outlined in FIG. 5. As shown in FIG. 5, the synthesis includes 5 steps. In particular, the synthesis includes a cyclization followed by an oxidation reaction. Subsequently, the product can undergo a protection reaction then the reduction of the ester. Finally, this is followed by an epoxidation resulting in the formation of the desired anti-epoxide intermediate.

In certain embodiments, the starting material can be tartaric acid. In certain embodiments, tartaric acid can undergo a cyclization reaction with an acid and acetone. In particular embodiments, the tartaric can undergo a reaction with the acid tosylic acid and acetone to afford the desired product. The reaction is illustrated in Scheme XVII.

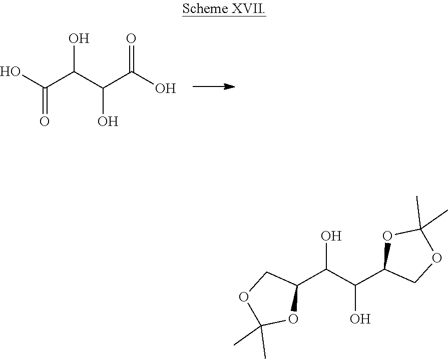

In certain embodiments, the resulting product can then be oxidized. In certain embodiments, the product is treated with an oxidizing agent. In particular embodiments, the product is treated with the oxidizing agent sodium periodate to afford the desired aldehyde. The reaction is illustrated in Scheme XVIII.

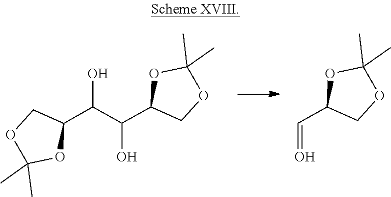

In certain embodiments, the resulting product undergo a reaction with protected 4-bromocatechol to afford the desired secondary alcohol. The reaction is illustrated in Scheme XIX.

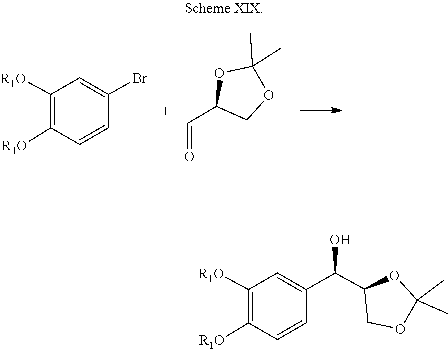

In certain embodiments, the resulting product can further undergo a ring opening followed by a sulfonylation reaction. In certain embodiments, the ring opening reaction can be accomplished using an acid. In particular embodiments, the acid is tosylic acid. In certain embodiments, the sulfonylation reaction can proceed by treating the resulting product with a mesyl chloride, tosyl chloride, or trifyl chloride in the presence of at least one catalyst and a base. In particular embodiments, the compound is treated with tosyl chloride to afford the sulfonate. The sulfonation reaction is illustrated in Scheme XX.

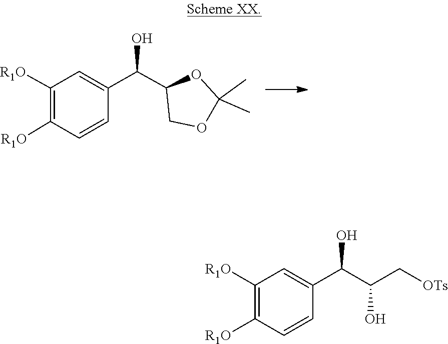

In certain embodiments, the resulting compound undergoes an epoxidation reaction. In certain embodiments, the compound is treated with a base. In certain embodiments, the base is sodium hydroxide, sodium hydride, or potassium hydroxide. In particular embodiments, the compound is treated with sodium hydroxide to afford the desired anti-epoxide. The epoxidation is illustrated in Scheme XXI.

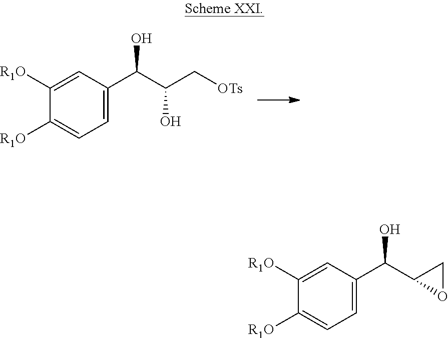

In certain embodiments, the resulting compound is further protected using one or more silyl protecting groups commonly known in the literature, including triispropylsilyl ether, t-butyldiphenylsilyl ether, or t-butyldimethylsilyl ether. In particular embodiments, the protecting group is t-butyldimethylsilyl ether. In certain embodiments, t-butyldimethylsilyl chloride is used to protect the hydroxy groups of the resulting compound. The protection reaction is illustrated in Scheme XXII.

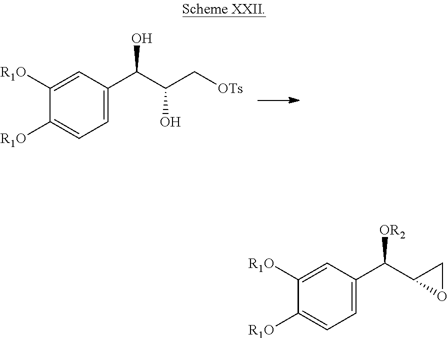

### Phase 2: Synthesis of (−)-Epicatechin

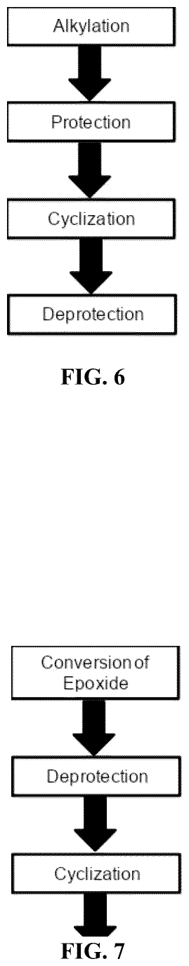

In certain embodiments, the second phase includes synthesis of (−)-epicatechin. In particular embodiments, (−)-epicatechin can be synthesized from the syn-epoxide. In particular embodiments, (−)-epicatechin can be synthesized from the anti-epoxide. In certain embodiments, the synthesis of the (−)-epicatechin proceeds following the general steps as outlined in FIGS. 6 and 7 and further discussed below.

Phase 2.1: Synthesis of (−)-Epicatechin from Syn-Epoxide Intermediate

In certain embodiments, the general synthesis of (−)-epicatechin from the syn-epoxide intermediate is outlined in FIG. 6. As shown in FIG. 6, the synthesis includes at least 5 steps. In particular, the synthesis includes an epoxide ring opening reaction. The product subsequently undergoes a protection followed by a deprotection reaction. The resulting product then undergoes a cyclization reaction. Finally, one or more deprotection reactions yields the desired (−)-epicatechin product.

In certain embodiments, the starting material for synthesis of (−)-epicatechin from the syn-epoxide is a compound of Formula VII where X is F, Br, I, Cl, N, S, or B and R₃ is Bn or MOM.

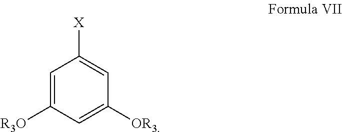

In certain embodiments, the syn-epoxide intermediate undergoes an epoxide ring opening reaction. In certain embodiments, the compound of Formula VII is 1,3-bis(benzyloxy)-5-fluorobenzene where X is F and R₃ is benzyl. In particular embodiments, the compound of Formula VI is treated with an organolithium reagent. In certain embodiments, the organolithium reagent is n-butyllithium, methyllithium, or tert-butyllithium. In particular embodiments, the organolithium reagents is n-butyllithium. In certain embodiments, the syn-epoxide intermediate is further added to the reaction mixture to afford the desired product. In alternative embodiments, the syn-epoxide intermediate can undergo an epoxide ring opening with the compound of Formula VI where X is Br, I, Cl, N, S, or B in the presence of a Lewis Acid known in the literature. The epoxide ring opening is illustrated in Scheme XXIII.

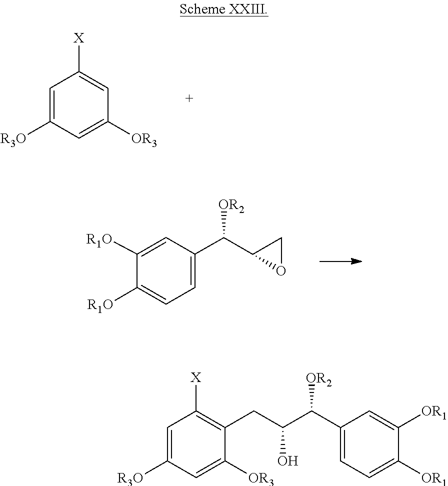

In certain embodiments, the resulting compound is further protected using one or more protecting groups commonly known in the literature, including methoxymethyl ether, tetrahydropyranyl ether, t-butyl ether, allyl ether, benzyl ether, triispropylsilyl ether or t-butyldimethylsilyl ether. In certain embodiments, the protecting group is different from the group at R₂. In certain particular embodiments, the protecting group is t-methoxymethyl. In certain embodiments, methoxymethyl chloride is used to protect the hydroxy groups of the resulting compound. The protection reaction is illustrated in Scheme XXIV.

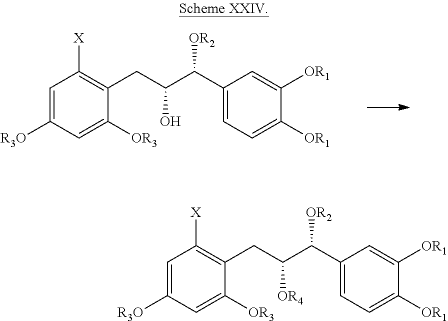

In certain embodiments, the protected compound further undergoes selective deprotection of the protecting group at R₂. In particular embodiments, R₂ is t-butyldimethylsilyl. In, certain embodiments, the protected compound is treated with an appropriate acid or fluoride known in the literature for deprotection of silyl ethers. In particular embodiments, the protected compound is treated with tetrabutylammonium fluoride (TBAF) to afford the desired deprotected compound. The deprotection reaction is illustrated in Scheme XXV.

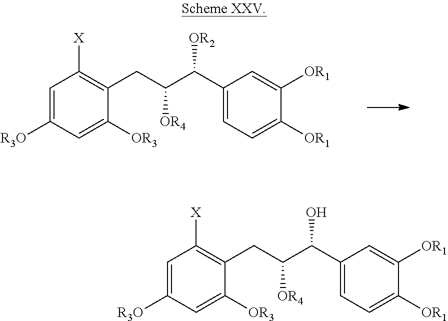

In certain embodiments, the resulting product is subjected to a cyclization reaction. In certain embodiments, the resulting product is treated with a strong base. In certain embodiments, the strong base is potassium hydride, sodium hydride, or potassium tert-butoxide. In particular embodiments, X is F and the strong base is potassium hydride to afford the desired cyclized product. The deprotection reaction is illustrated in Scheme XXVI.

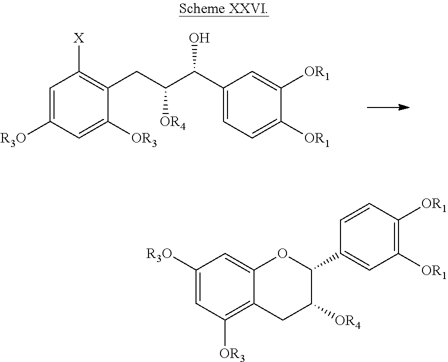

In alternative embodiments, where X is Br, I, Cl, N, S, or B the cyclization reaction is carried out in the presence of a copper catalyst to afford the desired cyclized product.

In certain embodiments, the cyclized product further undergoes one or more deprotection reactions. In certain embodiments, a methoxymethyl ether group is removed using a method known in the literature. In certain embodiments, the methoxymethyl ether group is removed using an acid. In particular embodiments, the acid is hydrochloric acid.

In certain embodiments, a benzyl ether group is removed using a debenzylation reaction. In certain embodiments, a palladium catalyst in the presence of hydrogen gas is used for debenzylation. In certain embodiments, the palladium catalyst can be palladium on carbon (Pd/C) or palladium hydroxide on carbon (Pd(OH)₂/C). In particular embodiments, the palladium catalyst is Pd(OH)₂/C in the presence of hydrogen gas. In particular embodiments, the one or more deprotection reactions afford the desired (−)-epicatechin product. The deprotection reactions are illustrated in Scheme XXVII.

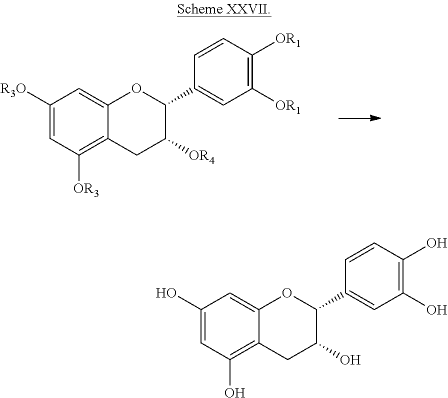

Phase 2.2: Synthesis of (−)-Epicatechin from Anti-Epoxide Intermediate

In certain embodiments, the general synthesis of (−)-epicatechin from the anti-epoxide intermediate is outlined in FIG. 7. As shown in FIG. 7, the synthesis includes at least 5 steps. In particular, the synthesis includes a first reaction. The product subsequently undergoes a protection followed by a deprotection reaction. The resulting product then undergoes a cyclization reaction. Finally, a deprotection reactions yields the desired (−)-epicatechin product.

In certain embodiments, the anti-epoxide undergoes a first reaction. The conversion is illustrated in Scheme XXVIII.

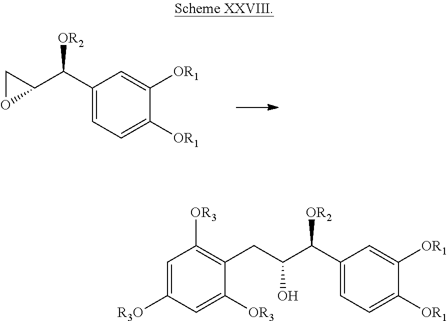

In certain embodiments, the resulting compound further undergoes one or more deprotection reactions. In particular embodiments, R₂ is t-butyldimethylsilyl. In, certain embodiments, the protected compound is treated with an appropriate acid or fluoride known in the literature for deprotection of silyl ethers. In particular embodiments, the protected compound is treated with tetrabutylammonium fluoride (TBAF) to afford the desired deprotected compound.

In certain embodiments, R₁ and R₃ are different. In particular embodiments, R₁ is a benzyl and R₃ is a methoxymethyl group. In certain embodiments, a methoxymethyl ether group is removed using a method known in the literature. In certain embodiments, the methoxymethyl ether group is removed using an acid. In particular embodiments, the acid is hydrochloric acid. The deprotection reaction is illustrated in Scheme XXIX.

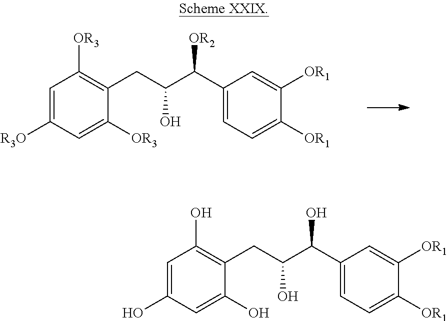

In certain embodiments, the resulting product is subjected to a cyclization reaction. In certain embodiments, the resulting product is treated with triethyl orthopropionate. In certain embodiments, the reaction mixture is further treated with an acid. In particular embodiments, the reaction mixture is treated with tosylic acid to afford the desired cyclized product. The cyclization reaction is illustrated in Scheme XXX.

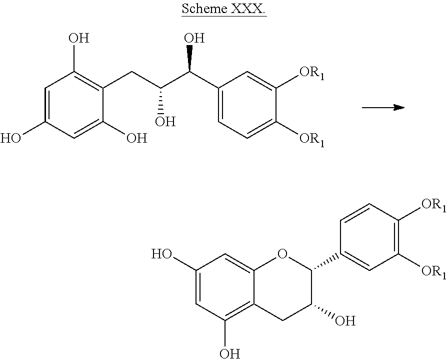

In certain embodiments, the resulting product undergoes a deprotection reaction. In particular embodiments, a benzyl ether group is removed using a debenzylation reaction. In certain embodiments, a palladium catalyst in the presence of hydrogen gas is used for debenzylation. In certain embodiments, the palladium catalyst can be palladium on carbon (Pd/C) or palladium hydroxide on carbon (Pd(OH)₂/C). In particular embodiments, the palladium catalyst is Pd(OH)₂/C in the presence of hydrogen gas. In particular embodiments, the deprotection reaction affords the desired (−)-epicatechin product. The deprotection reactions are illustrated in Scheme XXXI.

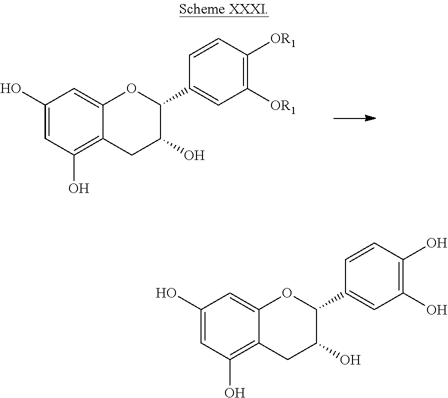

### IV. Consumer Products

In certain embodiments, the present disclosure further relates to consumer products including one or more catechin epimers disclosure herein. In certain embodiments, the consumer products include (−)-epicatechin, wherein each of the compounds has been prepared by methods disclosed herein.

In certain embodiments, the stereospecific catechin epimers provided herein can be used in a wide variety of edible products. Non-limiting examples of suitable food products include chocolates, chewing gum compositions, hard and soft confectionery products, dairy products, snack food products, food products of the beverage category where the product is at about a neutral pH, food products of the frozen food category, including frozen dairy products, nutritional products, nutritional supplements and pharmaceuticals and food categories described herein.

As used herein, “beverage category” can refer to beverages, beverage mixes and concentrates, including but not limited to, alcoholic and non-alcoholic ready to drink and dry powdered beverages, where the beverage is at about a neutral pH. Additional non-limiting examples of beverages can include carbonated and non-carbonated beverages, e.g., sodas, fruit or vegetable juices.

As used herein, “frozen food category” refers to chilled or frozen food products that have a neutral pH. Non-limiting examples of food products of the frozen food category can include ice cream, impulse ice cream, single portion dairy ice cream, single portion water ice cream, multi-pack dairy ice cream, multi-pack water ice cream, take-home ice cream, take-home dairy ice cream, ice cream desserts, bulk ice cream, take-home water ice cream, frozen yogurt, artisanal ice cream, frozen ready meals, frozen pizza, chilled pizza, frozen soup, frozen pasta, frozen processed red meat, frozen processed poultry, frozen processed fish/seafood, frozen vegetables, frozen processed vegetables, frozen meat substitutes, frozen potatoes, frozen bakery products and frozen desserts.

As used herein, “snack food category” refers to any food that can be a light informal meal including, but not limited to sweet and savory snacks and snack bars, where the foods have a neutral pH. Examples of snack foods include, but are not limited to, fruit snacks, chips/crisps, extruded snacks, tortilla/corn chips, popcorn, pretzels, nuts and other sweet and savory snacks. Examples of snack bars include, but are not limited to granola/muesli bars, breakfast bars, energy bars, fruit bars and other snack bars.

As used herein, “nutritional products” refers any product that can impart a desired nutritional value including, but not limited to, nutritional bars and nutritional beverages. In certain embodiments, the nutritional products are medical foods and/or adjunct nutritional therapies. Non-limiting examples of nutritional bars include protein bars (e.g., RXBAR®, LARABAR®, CLIF BAR®), nut bars (e.g., KIND® bars), energy bars, fiber bars, meal replacement bars, or other nutritional bars. Non-limiting examples of nutritional beverages include protein drinks (e.g., BOOST® Drinks), meal replacement shakes and/or drinks (e.g., Ensure® Nutritional Shakes), diabetes shakes and/or drinks (e.g., Glucerna® Shakes), immune support drinks, pediatric supplement drinks, or other nutritional beverages.

As used herein, “nutritional supplements” refers a supplement that can imparts a desired nutrition. Nutritional supplements can be, for example, administered orally in the form of a pill, chew, gummy, or powder. Non-limiting examples of nutritional supplements include vitamin supplements, mineral supplements, botanical supplements, other nutritional supplements, or combinations thereof. As used herein, “pharmaceutical” refers to a medical drug. Non-limiting examples of pharmaceuticals include prescription drugs, over-the-counter drugs (e.g. aspirin, acetaminophen, or ibuprofen), botanical drugs, or other pharmaceuticals.

In certain embodiments, effective amounts of (−)-epicatechin can be added to consumer products that do not naturally contain flavanols to provide desired health benefits to consumers. These amounts can be determined from existing, ongoing clinical trials. The flavanol monomers can also be added to foods and beverages that already contain flavanols to enrich the products further to provide improved health benefits.

Pharmaceuticals containing (−)-epicatechin of the present disclosure can be administered in several ways such as orally, nasally, buccally, intravenously, and topically. A person of skill in the art would be able to determine the appropriate mode of delivery as to maximize the effectiveness of the catechin compounds.

### EXAMPLES

The presently disclosed subject matter will be better understood by reference to the following Example, which is provided as exemplary of the presently disclosed subject matter, and not by way of limitation.

### Example 1: Synthesis of (−)-epicatechin

Example 1 provides the synthesis of (−)-epicatechin in twelve steps. Chiral auxiliary 1 and 3,4-bis(methoxymethoxy)benzaldehyde 2 are used as starting materials. The steps of this synthesis are shown in Scheme XXXII and further described below.

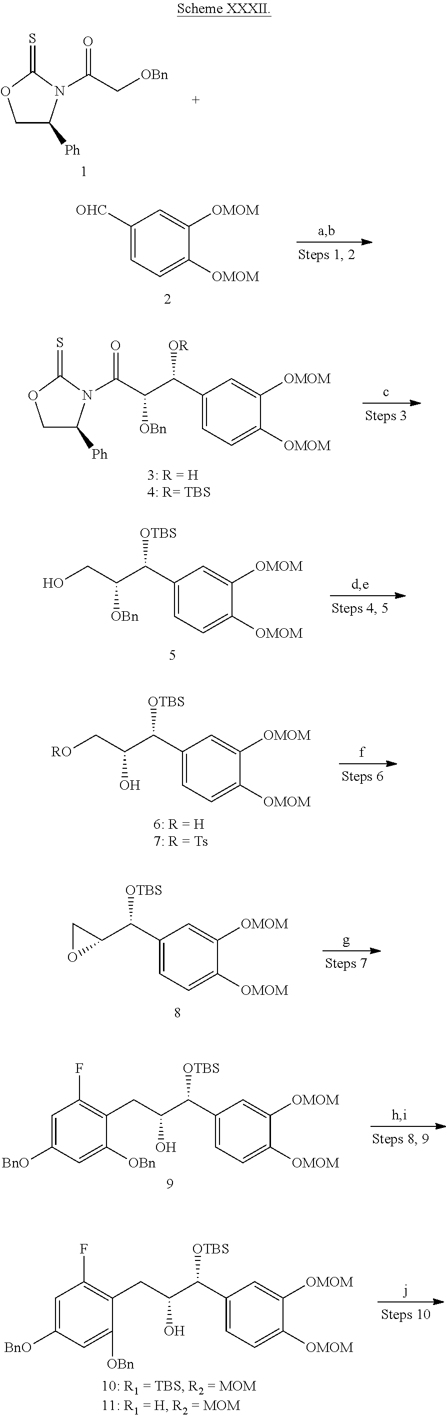

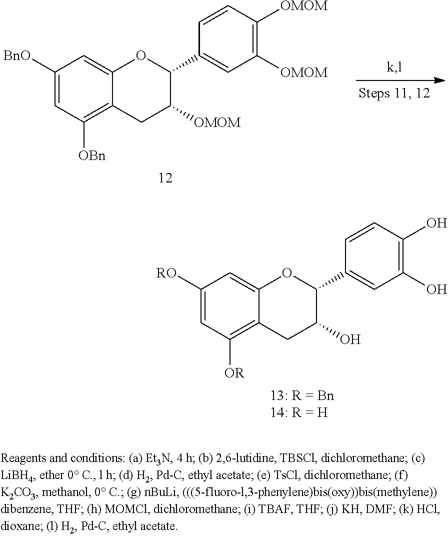

The chiral auxiliary 1 and 3,4-bis(methoxymethoxy)benzaldehyde 2 was subjected to an asymmetric aldol reaction using titanium tetrachloride and triethylamine to afford the desired product, (2S,3R)-2-(benzyloxy)-3-(3,4-bis(methoxymethoxy)phenyl)-3-hydroxy-1-((S)-4-phen-yl-2-thioxooxazolidin-3-yl)propan-1-one 3. The product then underwent a protection reaction with t-butyldimethylsilyl chloride (TBSCl) to give the desired t-butyldimethylsilyl ether, (2S,3R)-2-(benzyloxy)-3-(3,4-bis(methoxymethoxy)phenyl)-3-((tert-butyldimethylsilyl)oxy)-1-((S)-4-phen-yl-2-thioxooxazolidin-3-yl)propan-1-one 4. The chiral auxiliary was removed in a reduction reaction in the presence of lithium borohydride (LiBH₄) at 0° C. to produce (2R,3R)-2-(benzyloxy)-3-(3,4-bis(methoxymethoxy)phenyl)-3-((tert-butyldimethylsilyl)oxy)propan-1-ol 5. The benzyl group was then removed with Pd/C under H₂ via hydrogenation gives to give the desired alcohol, (2R,3R)-3-(3,4-bis(methoxymethoxy)phenyl)-3-((tert-butyldimethylsilyl)oxy)propane-1,2-diol 6. The alcohol 6 was then subjected to selective tosylation of the primary alcohol gives to give the desired tosylate, (2R,3R)-3-(3,4-bis(methoxymethoxy)phenyl)-3-((tert-butyldimethylsilyl)oxy)-2-hydroxypropyl 4-methylbenzenesulfonate 7. The tosylate 7 was further treated with potassium carbonate in methanol to give the desired epoxide, ((R)-(3,4-bis(methoxymethoxy)phenyl)((R)-oxiran-2-yl)methoxy)(tert-butyl)dimethylsilane 8. The epoxide 8 then underwent alkylation with (((5-fluoro-1,3-phenylene)bis(oxy))bis(methylene))dibenzene using n-butyl lithium in THF to give the desired alcohol, (1R,2R)-3-(2,4-Bis(benzyloxy)-6-fluorophenyl)-1-(3,4-bis(methoxymethoxy)phenyl)-1-((tert-butyldimethylsilyl)oxy)propan-2-ol 9. The free hydroxyl group of the alcohol 9 was then protected using chloromethylmethoxy ether (MOMCl) to afford the desired product (5R,6R)-5-{[2,4-Bis(benzyloxy)-6-fluorophenyl]methyl}-6-[3,4-bis(methoxymethoxy)phenyl]-8,8,9,9-tetramethyl-2,4,7-trioxa-8-siladecane 10. The desired product was then deprotected with tetrabutylammonium fluoride (TBAF) to give the desired alcohol, (1R,2R)-3-(2,4-bis(benzyloxy)-6-fluorophenyl)-1-(3,4-bis(methoxymethoxy)phenyl)-2-(methoxymethoxy)propan-1-ol 11. The alcohol 11 was then subjected to potassium hydride to give the desired protected (−)-epicatechin, (2R,3R)-5,7-bis(benzyloxy)-2-(3,4-bis(methoxymethoxy)phenyl)-3-(methoxymethoxy)chromane 12. Finally, deprotection of the methoxymethyl ethers with HCl to give the selectively deprotected product, 4-((2R,3R)-5,7-bis(benzyloxy)-3-hydroxychroman-2-yl)benzene-1,2-diol 13. This deprotection was followed by the deprotection the benzyl ether with Pd/C under H₂ gas to give (−)-epicatechin 14 as an off-white solid

### Example 2: Synthesis of (S)-2-(Benzyloxy)-1-(4-phenyl-2-thioxooxazolidin-3-yl)ethan-1-one (1)

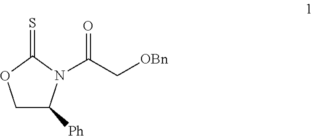 [SMILES: O=C(COCc1ccccc1)N1C(=S)OC[C@@H]1c1ccccc1]

While under nitrogen, a solution of (4S)-4-phenyl-1,3-oxazolidine-2-thione (1 eq., 5.5 g, 30.69 mmol) benzyloxyacetic acid (1 eq., 5.099 g, 4.39 mL, 30.69 mmol), and DMAP (0.25 eq., 0.94 g, 7.67 mmol) in methylene chloride (100 mL) was cooled to 0° C. and treated with EDCI (1.1 eq., 5.24 g, 33.75 mmol). After stirring for 4 h at 0° C., the ice-bath was removed and stirring was continued for 16 h. The reaction mixture was diluted with methylene chloride (100 mL) and washed with water (2×50 mL). The separated organic layer was washed successively with 1 N aq. HCl (50 mL), water (50 mL), 1M aq. NaOH (2×50 mL), water (50 mL), brine (20 mL). The resulting organic phase was dried over sodium sulfate, filtered, and concentrated. The resulting crude solid was crystallized from a mixture of ethyl acetate and hexanes to afford 2-(benzyloxy)-1-[(4S)-4-phenyl-2-sulfanylidene-1,3-oxazolidin-3-yl]ethan-1-one (8.52 g, 26.023 mmol, 84.8%) as off-white needles. ¹H NMR (400 MHz, CDCl₃) δ 4.53 (dd, J=8, 4 Hz, 1H), 4.60 (s, 2H), 4.86 (t, J=8 Hz, 1H), 5.04 (dd, J=36, 16 Hz, 2H), 5.71 (dd, J=8, 4 Hz, 1H), 7.29-7.41 (m, 10H).

### Example 3: Synthesis of 3,4-bis(methoxymethoxy)benzaldehyde (2)

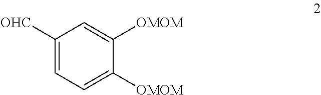

While under nitrogen, a stirred solution of 3,4-dihydroxybenzaldehyde (1 eq., 1 g, 7.24 mmol) in DMF (20 mL) was cooled to 0° C. (ice-bath) and treated with potassium carbonate (9 eq., 9.005 g, 65.16 mmol). After stirring for 15 min, chloromethyl methyl ether (4.5 eq., 2.62 g, 32.58 mmol) was added drop-wise. After the addition was complete, the thick slurry was stirred at room temperature overnight. The reaction mixture was filtered, and washed thoroughly with ethyl acetate. The filtrate was diluted with water (200 mL) and extracted with ethyl acetate (3×15 mL). The combined organic layer was washed with water (2×15 mL), dried over sodium sulfate and concentrated to get 3,4-bis(methoxymethoxy)benzaldehyde (1.5 g, 6.63 mmol, 91.58%) as a light brown oil. This material was used in the subsequent step without further purification.

### Example 4: Synthesis of (2S,3R)-2-(Benzyloxy)-3-(3,4-bis(methoxymethoxy)phenyl)-3-hydroxy-1-((S)-4-phenyl-2-thioxooxazolidin-3-yl)propan-1-one (3)

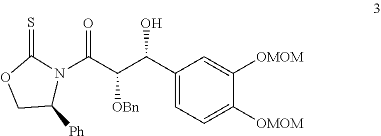

While under argon, a solution of 2-(benzyloxy)-1-[(4S)-4-phenyl-2-sulfanylidene-1,3-oxazolidin-3-yl]ethan-1-one (1 eq., 723 mg, 2.208 mmol) in anhydrous dichloromethane (20 mL) was cooled to −10° C., and treated with titanium tetrachloride (1.1 eq., 2.43 mL, 2.43 mmol) (1.0 M in dichloromethane) in a drop-wise manner. After stirring for 30 min, the solution was cooled to −78° C. After 5 min, triethylamine (2 eq., 446.93 mg, 0.61 mL, 4.42 mmol) was added in a drop-wise manner. The color of the solution turned deep purple from amber upon the addition of triethylamine. After stirring for 75 min, 3,4-bis(methoxymethoxy)benzaldehyde (2 eq., 999.16 mg, 4.42 mmol) in dichloromethane (5 mL) was added. After the addition was complete, stirring was continued at −78° C. for 2 h. Once complete, the mixture was diluted with brine (30 mL), and allowed to gently warm to 0° C. The quenched reaction mixture was extracted with 1M aq. HCl (2×30 mL), and water (30 mL). The organic layer was then dried over sodium sulfate, filtered and concentrated under reduced pressure. The residue was purified by silica gel flash column chromatography (20-40% ethyl acetate in hexanes) to give (2S,3R)-2-(benzyloxy)-3-[3,4-bis(methoxymethoxy)phenyl]-3-hydroxy-1-[(4S)-4-phenyl-2-sulfanylidene-1,3-oxazolidin-3-yl]propan-1-one (760 mg, 1.37 mmol, 62.16%) as a light yellow foam. ¹H NMR (400 MHz, CDCl₃) δ 2.97 (brs, 1H), 3.52 (s, 6H), 4.29-4.35 (m, 2H), 4.51 (dd, J=12, 8 Hz, 2H), 4.98 (d, J=8 Hz, 1H), 5.19 (d, J=8 Hz, 1H), 5.21 (d, J=8 Hz, 1H), 5.26 (dd, J=4, 2 Hz, 2H), 5.33 (dd, J=4, 2 Hz, 1H), 6.50 (d, J=4 Hz, 1H), 7.12 (d, J=8 Hz, 1H), 7.14-7.24 (m, 7H), 7.31 (d, J=4 Hz, 1H), 7.34-7.41 (m, 3H).

### Example 5: Synthesis of (2S,3R)-2-(Benzyloxy)-3-(3,4-bis(methoxymethoxy)phenyl)-3-((tert-butyldimethylsilyl)oxy)-1-((S)-4-phenyl-2-thioxooxazolidin-3-yl)propan-1-one (4)

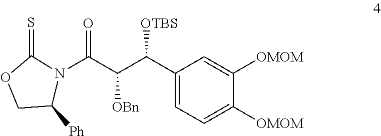

While under nitrogen, a stirred solution of 2,6-lutidine (2.4 eq., 353.038 mg, 0.38 mL, 3.29 mmol) in dichloromethane (10 mL) was cooled to −78° C. and charged with tert-butyldimethylsilyl trifluoromethanesulfonate (2.2 eq., 798.29 mg, 0.69 mL, 3.02 mmol) and (2S,3R)-2-(benzyloxy)-3-[3,4-bis(methoxymethoxy)phenyl]-3-hydroxy-1-[(4S)-4-phenyl-2-sulfanylidene-1,3-oxazolidin-3-yl]propan-1-one (1 eq., 760 mg, 1.37 mmol) in dichloromethane (5 mL), drop-wise. After stirring at −78° C. for 2 h, the reaction mixture was quenched with 50% sat. sodium bicarbonate, warmed to room temperature and diluted with dichloromethane (50 mL). The organic layer was separated, dried over sodium sulfate, filtered, and concentrated. The crude product was purified by silica gel column chromatography (10-20% ethyl acetate in hexanes) to give (2S,3R)-2-(benzyloxy)-3-[3,4-bis(methoxymethoxy)phenyl]-3-[(tert-butyldimethylsilyl)oxy]-1-[(4S)-4-phenyl-2-sulfanylidene-1,3-oxazolidin-3-yl]propan-1 (440 mg, 0.66 mmol) as a colorless oil. ¹H NMR (400 MHz, CDCl₃) δ −0.09 (s, 3H), 0.03 (s, 3H), 0.85 (s, 9H), 3.52 (s, 6H), 4.00 (t, J=8 Hz, 1H), 4.15 (dd, J=12, 4 Hz, 1H), 4.58 (d, J=12 Hz, 1H), 4.67 (d, J=12 Hz, 1H), 4.88 (d, J=4 Hz, 1H), 5.06 (dd, J=8, 4 Hz, 1H), 5.18 (t, J=8 Hz, 2H), 5.26 (d, J=8 Hz, 1H), 5.31 (d, J=4 Hz, 1H). 6.56 (d, J=8 Hz, 1H), 6.91 (dd, J=8, 2 Hz, 1H), 7.06 (d, J=8 Hz, 1H), 7.09-7.11 (m, 2H), 7.19-7.25 (m, 5H), 7.30-7.34 (m, 3H).

### Example 6: Synthesis of (2R,3R)-2-(Benzyloxy)-3-(3,4-bis(methoxymethoxy)phenyl)-3-((tert-butyldimethylsilyl)oxy)propan-1-ol (5)

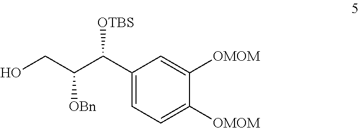

A solution of (2S,3R)-2-(benzyloxy)-3-[3,4-bis(methoxymethoxy)phenyl]-3-[(tert-butyldimethylsilyl)oxy]-1-[(4S)-4-phenyl-2-sulfanylidene-1,3-oxazolidin-3-yl]propan-1 (1 eq., 440 mg, 0.66 mmol) in diethyl ether (10 mL) and MeOH (2 mL) was cooled to 0° C. and treated with lithium borohydride (1.5 eq., 20.805 mg, 0.96 mmol) in one portion. After stirring for 1 h at 0° C., the mixture was quenched with a saturated aqueous NaHCO₃ solution. The mixture was extracted with ethyl acetate and hexanes (10:1) (3×15 mL). The combined extracts were dried over sodium sulfate, filtered and concentrated. The crude product was purified by silica gel flash column chromatography (15-30% ethyl acetate in hexanes) to give (2R,3R)-2-(benzyloxy)-3-[3,4-bis(methoxymethoxy)phenyl]-3-[(tert-butyldimethylsilyl)oxy]propan-1-ol (298 mg, 0.605 mmol, 91.81%) as a colorless oil. ¹H NMR (400 MHz, CDCl₃) δ −0.11 (s, 3H), 0.04 (s, 3H), 0.88 (s, 9H), 1.94 (t, J=8 Hz, 1H), 3.31 (dd, J=12, 8 Hz, 1H), 3.47-3.56 (m, 1H), 3.49 (s, 3H), 3.52 (s, 3H), 3.57-3.61 (m, 1H), 4.63 (d, J=12 Hz, 1H), 4.77 (d, J=8 Hz, 1H), 4.83 (d, J=12 Hz, 1H), 5.19-5.22 (m, 4H), 6.90 (dd, J=8, 4 Hz, 1H), 7.08 (d, J=8 Hz, 1H), 7.19 (d, J=4 Hz, 1H).

### Example 7. Synthesis of (2R,3R)-3-(3,4-Bis(methoxymethoxy)phenyl)-3-((tert-butyldimethylsilyl)oxy)propane-1,2-diol (6)

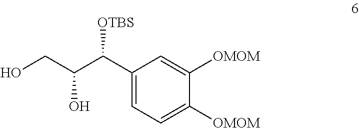

A heterogeneous solution of (2R,3R)-2-(benzyloxy)-3-[3,4-bis(methoxymethoxy)phenyl]-3-[(tert-butyldimethylsilyl)oxy]propan-1-ol (300 mg, 0.609 mmol) and palladium hydroxide (300 mg, 2.14 mmol) (20% on carbon) was evacuated and filled with hydrogen three times, then stirred under hydrogen atmosphere (balloon) for 15 h. The mixture was filtered and washed with ethyl acetate. The combined filtrate was concentrated under reduced pressure and purified by silica gel flash column chromatography (20-60% ethyl acetate in hexanes) to give (2R,3R)-3-[3,4-bis(methoxymethoxy)phenyl]-3-[(tert-butyldimethylsilyl)oxy]propane-1,2-diol (200 mg, 0.5 mmol, 81.59%) as a colorless, thick oil. ¹H NMR (400 MHz, CDCl₃) δ −0.15 (s, 3H), 0.05 (s, 3H), 0.90 (s, 9H), 1.95 (t, J=8 Hz, 1H), 2.82 (d, J=4 Hz, 1H), 3.43-3.53 (m, 1H), 3.50 (s, 3H), 3.52 (s, 3H), 3.56-3.64 (m, 2H), 4.60 (d, J=8 Hz, 1H), 5.21-5.24 (m, 4H), 6.89 (dd, J=8, 4 Hz, 1H), 7.09 (d, J=8 Hz, 1H), 7.16 (d, J=4 Hz, 1H).

### Example 8: Synthesis of (2R,3R)-3-(3,4-Bis(methoxymethoxy)phenyl)-3-((tert-butyldimethylsilyl)oxy)-2-hydroxypropyl 4-methylbenzenesulfonate (7)

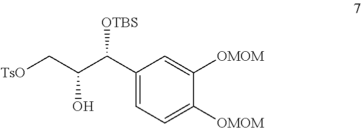

While under nitrogen, a stirred solution of (2R,3R)-3-[3,4-bis(methoxymethoxy)phenyl]-3-[(tert-butyldimethylsilyl)oxy]propane-1,2-diol (1 eq., 200 mg, 0.5 mmol) in anhydrous dichloromethane (4 mL) was cooled to 0° C. and treated with dibutyltin oxide (0.02 eq., 2.47 mg, 0.0099 mmol), p-toluenesulfonyl chloride (1.2 eq., 113.66 mg, 0.6 mmol), DMAP (0.1 eq., 6.07 mg, 0.05 mmol), and triethylamine (1.2 eq., 60.33 mg, 0.083 mL, 0.6 mmol). After stirring for 5 h, the reaction mixture was quenched with water (5 mL). The layers were separated, and aqueous layer was extracted with dichloromethane (2×10 mL). The combined organic layer was dried over sodium sulfate, filtered, and concentrated. The residue was purified by silica gel flash column chromatography (15-30% ethyl acetate in hexanes) to give [(1R,2R)-1-[3,4-bis(methoxymethoxy)phenyl]-2-hydroxy-3-[(4-methylbenzenesulfonyl)oxy]propoxy](tert-butyl)-dimethylsilane (260 mg, 0.47 mmol, 94%) as a colorless oil. ¹H NMR (400 MHz, CDCl₃) δ −0.17 (s, 3H), 0.02 (s, 3H), 0.86 (s, 9H), 2.44 (s, 3H), 2.60 (d, J=4 Hz, 1H), 3.49 (s, 3H), 3.52 (s, 3H), 3.72-3.76 (m, 1H), 3.84 (dd, J=12, 8 Hz, 1H), 4.02 (dd, J=12.4 Hz, 1H), 4.61 (d, J=4 Hz, 1H), 5.20-5.22 (m, 4H), 6.81 (dd, J=8, 4 Hz, 1H), 7.06 (d, J=8 Hz, 1H), 7.12 (d, J=4 Hz, 1H), 7.33 (d, J=8 Hz, 2H), 7.78 (d, J=8 Hz, 2H).

### Example 9: Synthesis of ((R)-(3,4-Bis(methoxymethoxy)phenyl)((R)-oxiran-2-yl)methoxy)(tert-butyl)dimethylsilane (8)

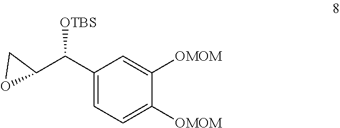

While under nitrogen, a solution of [(1R,2R)-1-[3,4-bis(methoxymethoxy)phenyl]-2-hydroxy-3-[(4-methylbenzenesulfonyl)oxy]propoxy](tert-butyl)dimethylsilane (1 eq., 260 mg, 0.47 mmol) in MeOH (2.5 mL) and dioxane (1 mL) was cooled to 0° C. (ice-bath) and treated with potassium carbonate (2 eq., 129.085 mg, 0.93 mmol). After stirring for 1.0 h at 0° C., the ice-bath was removed and stirring was continued for 2 h. Once complete, the reaction mixture was diluted with ethyl acetate (10 mL), filtered, and washing thoroughly with ethyl acetate. The filtrate was concentrated under reduced pressure and purified by silica gel flash column chromatography (20% ethyl acetate in hexanes) to give [(R)-[3,4-bis(methoxymethoxy)phenyl][(2R)-oxiran-2-yl]methoxy](tert-butyl)dimethylsilane (148 mg, 0.38 mmol, 82.41%) as a colorless oil. ¹H NMR (400 MHz, CDCl₃) δ 0.00 (s, 3H), 0.11 (s, 3H), 0.91 (s, 9H), 2.65 (dd, J=8, 4 Hz, 1H), 2.75 (t, J=8 Hz, 1H), 3.04-3.07 (m, 1H), 3.51 (s, 3H), 3.52 (3H), 4.29 (d, J=4 Hz, 1H), 5.21-5.23 (m, 4H), 6.90 (dd, J=8, 4 Hz, 1H), 7.11 (d, J=8 Hz, 1H), 7.22 (s, 1H).

### Example 10: Synthesis of (1R,2R)-3-(2,4-Bis(benzyloxy)-6-fluorophenyl)-1-(3,4-bis(methoxymethoxy)phenyl)-1-((tert-butyldimethylsilyl)oxy)propan-2-ol (9)

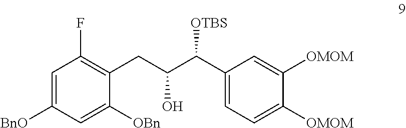

While under argon, a solution of 1,3-bis(benzyloxy)-5-fluorobenzene (2 eq., 232.54 mg, 0.75 mmol) in anhydrous THF (5 mL) was cooled to −78° C. was carefully treated with n-butyllithium (2.09 eq., 0.38 mL, 0.79 mmol, 2.3 M in hexanes) drop-wise. After stirring for 1 h, a solution of [(R)-[3,4-bis(methoxymethoxy)phenyl][(2R)-oxiran-2-yl]methoxy](tert-butyl)dimethylsilane (1 eq., 145 mg, 0.38 mmol) in anhydrous THF (2 mL) was slowly added, followed by a solution of boron trifluoride etherate (2 eq., 107.035 mg, 0.096 mL, 0.75 mmol) in anhydrous THF (2 mL). After stirring for 30 min, the reaction was quenched with MeOH (3 mL) and brine (10 mL). The mixture was extracted with ethyl acetate (3×15 mL) and the combined extracts were washed with brine (15 mL), dried over sodium sulfate, filtered and concentrated. The resulting residue was purified by silica gel flash column chromatography (5-15% ethyl acetate in hexanes) to give (1R,2R)-3-[2,4-bis(benzyloxy)-6-fluorophenyl]-1-[3,4-bis(methoxymethoxy)-phenyl]-1-[(tert-butyldimethylsilyl)oxy]propan-2-ol (125 mg, 0.18 mmol, 47.84%) as a colorless oil. ¹H NMR (400 MHz, CDCl₃) δ −0.16 (s, 3H), 0.06 (s, 3H), 0.89 (s, 9H), 2.50 (d, J=4 Hz, 1H), 2.51-2.72 (m, 2H), 3.47 (s, 3H), 3.52 (s, 3H), 3.82-3.89 (m, 1H), 4.50 (d, 4 Hz, 1H), 4.95 (s, 2H), 4.96 (s, 2H), 5.14 (s, 2H), 5.20 (s, 2H), 6.28-6.33 (m, 2H), 6.83 (dd, J=8, 2 Hz, 1H), 7.02 (d, J=8 Hz, 1H), 7.16 (d, J=2 Hz, 1H), 7.26-7.36 (m, 10 Hz). ¹⁹F NMR (376 MHz, CDCl₃) δ −114.52.

### Example 11: Synthesis of (5R,6R)-5-{[2,4-Bis(benzyloxy)-6-fluorophenyl]methyl}-6-[3,4-bis(methoxymethoxy)phenyl]-8,8,9,9-tetramethyl-2,4,7-trioxa-8-siladecane (10)

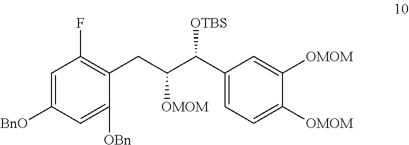

While under nitrogen, a solution of (1R,2R)-3-[2,4-bis(benzyloxy)-6-fluorophenyl]-1-[3,4-bis(methoxymethoxy)phenyl]-1-[(tert-butyldimethylsilyl)oxy]propan-2-ol (1 eq., 125 mg, 0.18 mmol), and diisopropylethyl amine (12 eq., 279.8 mg, 0.38 mL, 2.16 mmol) in dichloromethane (3 mL) was cooled to 0° C. and treated with chloromethyl methyl ether (6 eq., 87.15 mg, 0.082 mL, 1.082 mmol) and tetrabutylammonium iodide (0.05 eq., 3.33 mg, Stirring was continued at room temperature overnight. Once complete, the reaction mixture was poured into a saturated aqueous sodium bicarbonate solution (10 mL) and extracted with ethyl acetate (3×10 mL). The combined organic layer was dried over sodium sulfate, filtered and concentrated. The crude product was purified by silica gel flash column chromatography (5-15% ethyl acetate in hexanes) to give (5R,6R)-5-{[2,4-bis(benzyloxy)-6-fluorophenyl]methyl}-6-[3,4-bis(methoxymethoxy)phenyl]-8,8,9,9-tetramethyl-2,4,7-trioxa-8-siladecane (110 mg, 0.15 mmol, 82.74%). ¹H NMR (400 MHz, CDCl₃) δ −0.13 (s, 3H), 0.040 (s, 3H), 0.87 (s, 9H), 2.50 (d, J=8 Hz, 1H), 2.68 (dd, J=8, 4 Hz, 1H), 3.46 (s, 3H), 3.51 (s, 3H), 4.02-4.07 (m, 1H), 4.35 (d, J=8 Hz, 1H), 4.61 (dd, J=12, 8 Hz, 2H), 4.95 (s, 2H), 4.98 (s, 2H), 5.15 (s, 2H), 5.20 (s, 2H), 6.26 (d, J=12 Hz, 1H), 6.31 (brs, 1H), 6.83 (d, J=8 Hz, 1H), 7.02 (d, 8 Hz, 1H), 7.13 (s, 1H), 7.30-7.43 (m, 10H). ¹⁹F NMR (376 MHz, CDCl₃) δ −114.08.

### Example 12: Synthesis of (1R,2R)-3-(2,4-Bis(benzyloxy)-6-fluorophenyl)-1-(3,4-bis(methoxymethoxy)phenyl)-2-(methoxymethoxy)propan-1-ol (11)

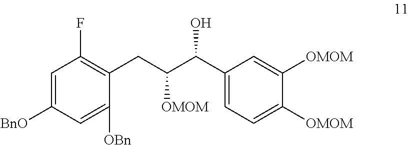

A stirred solution of (5R,6R)-5-{[2,4-bis(benzyloxy)-6-fluorophenyl]methyl}-6-[3,4-bis(methoxymethoxy)phenyl]-8,8,9,9-tetramethyl-2,4,7-trioxa-8-siladecane (1 eq., 110 mg, 0.15 mmol) in THF (3 mL) was cooled to 0° C. (ice-bath) and treated with tetrabutylammonium fluoride (1.5 eq., 0.22 mL, 0.22 mmol) (1.0 M in THF). The reaction mixture was stirred at room temperature, concentrated and purified by silica gel flash column chromatography (15-40% ethyl acetate in hexanes) to give (1R,2R)-3-[2,4-bis(benzyloxy)-6-fluorophenyl]-1-[3,4-bis(methoxymethoxy)phenyl]-2-(methoxymethoxy)propan-1-ol (90 mg, 0.14 mmol, 96.83%) as a colorless oil. ¹H NMR (400 MHz, CDCl₃) δ 2.72 (dd, J=16, 8 Hz, 1H), 2.88 (dd, J=16, 8 Hz, 1H), 3.09 (s, 3H), 3.47 (s, 3H), 3.49 (s, 3H), 3.35 (d, J=4 Hz, 1H), 3.94-3.99 (m, 1H), 4.37 (dd, J=12, 8 Hz, 2H), 4.49 (t, J=4 Hz, 1H), 4.97-5.00 (m, 4H), 5.15-5.20 (m, 4H), 6.32 (dd, J=12, 4 Hz, 1H), 6.37 (brs, 1H), 6.85 (dd, J=8, 4 Hz, 1H), 7.02 (d, J=8 Hz, 1H), 7.14 (d, J=4 Hz, 1H), 7.32-7.41 (m, 10H). ¹⁹F NMR (376 MHz, CDCl₃) δ −114.07.

### Example 13: Synthesis of (2R,3R)-5,7-Bis(benzyloxy)-2-(3,4-bis(methoxymethoxy)phenyl)-3-(methoxymethoxy)chromane (12)

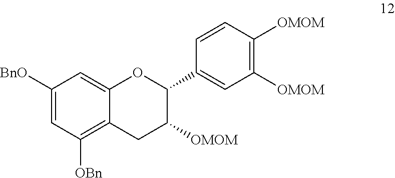

While under argon, a stirred solution of (1R,2R)-3-[2,4-bis(benzyloxy)-6-fluorophenyl]-1-[3,4-bis(methoxymethoxy)phenyl]-2-(methoxymethoxy)propan-1-ol (1 eq., 90 mg, 0.14 mmol) in anhydrous DMF (3 mL) was cooled to 0° C. (ice-bath) and treated with potassium hydride (5 eq., 96.61 mg, 0.72 mmol, 30% in paraffin oil). Once the addition was complete, the ice-bath was removed. After stirring for 2 h, the reaction re-cooled to 0° C. and quenched with water (30 mL). and extracted with ethyl acetate (3×10 mL). The organic layer was washed with water (10 mL), dried over sodium sulfate, filtered and concentrated. The crude product was purified by silica gel flash column chromatography (15-40% ethyl acetate in hexanes) to give (2R,3R)-5,7-bis(benzyloxy)-2-[3,4-bis(methoxymethoxy)phenyl]-3-(methoxymethoxy)-3,4-dihydro-2H-1-benzopyran (70 mg, 0.12 mmol, 80.36%) as a white solid. ¹H NMR (400 MHz, CDCl₃) δ 2.84 (dd, J=17.3, 4 Hz, 1H), 2.94 (s, 3H), 3.03 (dd, J=17.3, 4 Hz, 1H), 4.22 (brs, 1H), 4.30 (d, J=7 Hz, 1H), 4.61 (d, J=7 Hz, 1H), 4.98 (s, 3H), 5.01 (s, 2H), 5.21-5.25 (m, 4H), 6.23 (d, J=2.3 Hz, 1H), 6.28 (d, J=2.3 Hz, 1H), 7.09 (dd, J=8.5, 2 Hz, 1H), 7.15 (d, J=8.4 Hz, 1H), 7.28-7.41 (m, 10H).

### Example 14: Synthesis of 4-((2R,3R)-5,7-Bis(benzyloxy)-3-hydroxychroman-2-yl)benzene-1,2-diol (13)

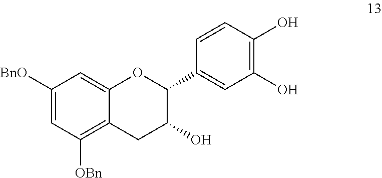 [SMILES: Oc1ccc([C@H]2Oc3cc(OCc4ccccc4)cc(OCc4ccccc4)c3C[C@H]2O)cc1O]

A solution of (2R,3R)-5,7-bis(benzyloxy)-2-[3,4-bis(methoxymethoxy)phenyl]-3-(methoxymethoxy)-3,4-dihydro-2H-1-benzopyran (1 eq., 70 mg, 0.12 mmol) in MeOH (2 mL) and dichloromethane (2 mL) was cooled to 0° C. and treated with HCl in dioxane (68.88 eq., 2 mL, 8 mmol). After 1 h, the ice-bath was removed and stirring was continued 1 h. The solvent was removed under reduced pressure and the residue was purified by silica gel flash chromatography (50-100% ethyl acetate in hexanes) to give 4-[(2R,3R)-5,7-bis(benzyloxy)-3-hydroxy-3,4-dihydro-2H-1-benzopyran-2-yl]benzene-1,2-diol (28 mg, 0.06 mmol, 51.24%) as a colorless solid. ¹H NMR (400 MHz, CDCl₃) δ 2.94 (dd, J=16, 4 Hz, 1H), 3.02 (d, J=16 Hz, 1H), 4.25 (brs, 1H), 4.92 (s, 1H), 5.00 (s, 2H), 5.02 (s, 2H), 6.27 (s, 2H), 6.87-6.90 (m, 2H), 7.07 (s, 1H), 7.30-7.41 (m, 10H).

### Example 15: Synthesis of (−)-Epicatechin

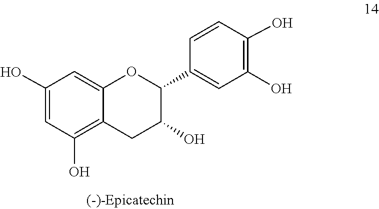 [SMILES: Oc1cc(O)c2c(c1)O[C@H](c1ccc(O)c(O)c1)[C@H](O)C2]

A solution of 4-[(2R,3R)-5,7-bis(benzyloxy)-3-hydroxy-3,4-dihydro-2H-1-benzopyran-2-yl]benzene-1,2-diol (1 eq., 25 mg, 0.053 mmol) in THF-MeOH-water (1:1:1) (3 mL) and Palladium hydroxide on carbon (4.021 eq., 30 mg, 0.21 mmol) (20% on carbon) was evacuated and filled with hydrogen (3 times). This heterogeneous solution was stirred overnight at room temperature under hydrogen atmosphere (balloon). The catalyst was removed by filtration and the filtrate was concentrated. Then the aq. residue was lyophilized to give (2R,3R)-2-(3,4-dihydroxyphenyl)-3,4-dihydro-2H-1-benzopyran-3,5,7-triol (9 mg, 0.031 mmol, 58.35%).

### Example 16: Alternate Synthesis of Syn-Epoxide Intermediate

The compound 3,4-bis(benzyloxymethoxy)benzaldehyde 15 can be used as a starting material to arrive at the syn-epoxide intermediate. The steps of this synthesis are shown in Scheme XXXIII and further described below.

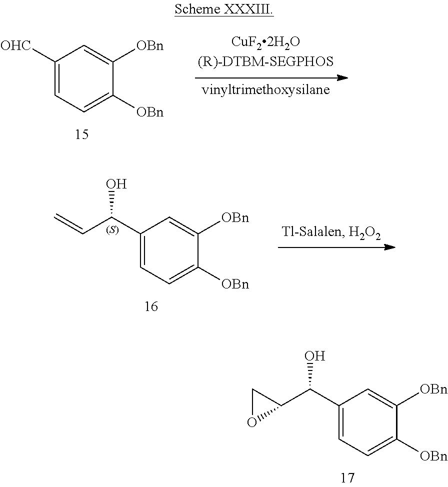

The compound 3,4-bis(benzyloxymethoxy)benzaldehyde 15 can undergo asymmetric addition of vinyl trimethoxysilane in the presence of the catalysts CuF-2H₂O and (R)-DTBM-SEGPHOS to afford the desired alkene, (S)-1-(3,4-bis(benzyloxy)phenyl)prop-2-en-1-ol 16. The alkene 16 can then further undergo epoxidation with titanium-salalen with hydrogen peroxide. Further steps can be the same as Steps 7-12 in Scheme XXXII from Example 1.

### Example 17: Alternate Synthesis of (−)-Epicatechin with Anti-Epoxide Intermediate

Example 17 provides the synthesis of (−)-epicatechin in seven steps. The compound 3,4-bis(benzyloxymethoxy)benzaldehyde 15 can be used as a starting materials. The steps of this synthesis are shown in Scheme XXXIV and further described below.

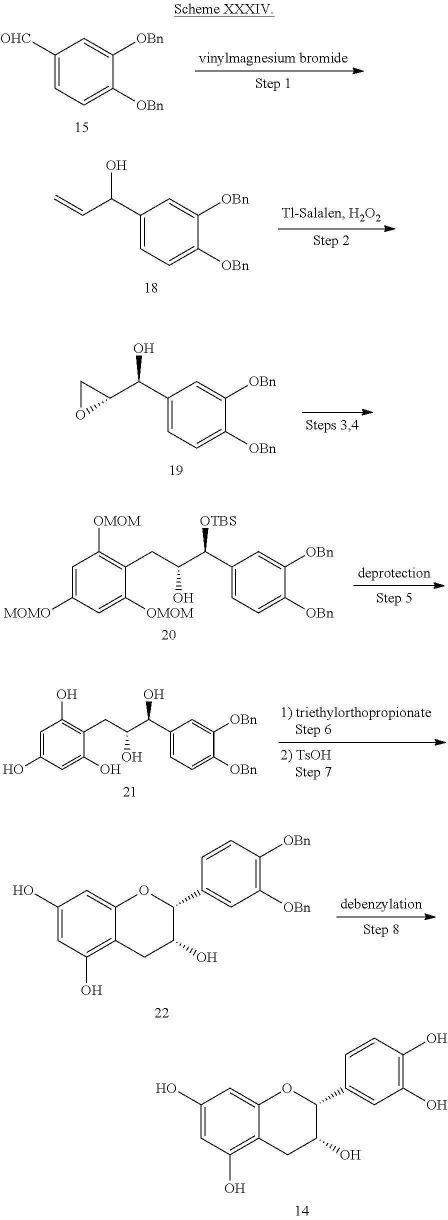

The compound 3,4-bis(benzyloxymethoxy)benzaldehyde 15 can undergo a Grignard reaction with the Grignard reagent vinylmagnesium bromide to afford the desired alkene, (S)-1 1-(3,4-bis(benzyloxy)phenyl)prop-2-en-1-ol 18. The alkene 18 can then further undergo epoxidation with titanium isopropoxide (Ti(OⁱPr)₄) and t-butyl hydroperoxide (TBHP) to give the desired epoxide, (S)-(3,4-bis(benzyloxy)phenyl)((R)-oxiran-2-yl)methanol 19. The epoxide 19 can then be converted to the product, (1S,2R)-1-(3,4-bis(benzyloxy)phenyl)-1-((tert-butyldimethylsilyl)oxy)-3-(2,4,6-tris(methoxymethoxy)phenyl)propan-2-ol 20. Product 20 can then undergo a deprotection to remove the t-butyldimethyl ether using deprotection conditions to give the desired alcohol, 2-((2R,3S)-3-(3,4-bis(benzyloxy)phenyl)-2,3-dihydroxypropyl)benzene-1,3,5-triol 21. The alcohol 21 can then undergo cyclization with triethyl orthopropionate and tosylation with tosylic acid to give the protected (−)-epicatechin, 2-((2R,3S)-3-(3,4-bis(benzyloxy)phenyl)-2,3-dihydroxypropyl)-benzene-1,3,5-triol 22. The protected product 22 can then be deprotected via a debenzylation reaction to afford the desired (−)-epicatechin 14.

### Example 18: Alternate Synthesis of Anti-Epoxide Intermediate

The compound 3,4-bis(benzyloxymethoxy)benzaldehyde 15 can be used as a starting material to arrive at the desired anti-epoxide intermediate. The steps of this synthesis are shown in Scheme XXXV and further described below.

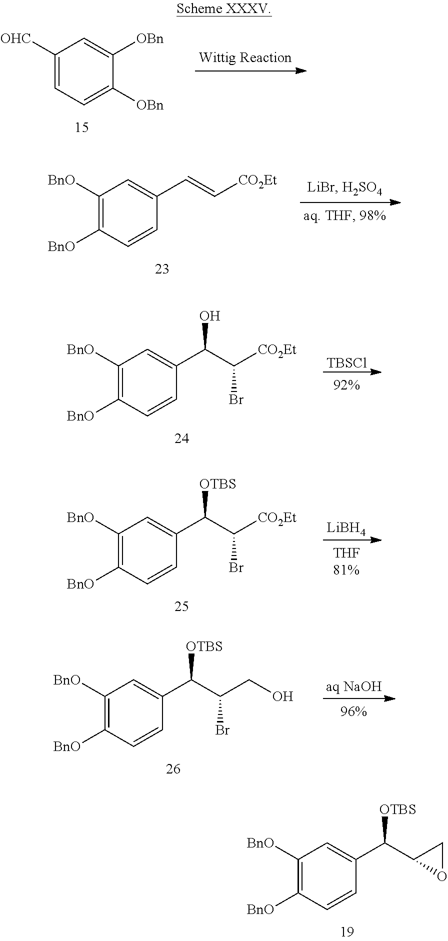

The compound 3,4-bis(benzyloxymethoxy)benzaldehyde 15 can undergo a Wittig reaction with the appropriate ylide to give the desired alkene, ethyl (E)-3-(3,4-bis(benzyloxy)phenyl)acrylate 23. The alkene 23 can then undergo an addition reaction to arrive at the desired alcohol, ethyl (2R,3R)-3-(3,4-bis(benzyloxy)phenyl)-2-bromo-3-hydroxypropanoate 24. The desired alcohol 24 can be protected with a t-butyldimethyl silyl ether using TBSCl to give the desired protected product, ethyl (2R,3R)-3-(3,4-bis(benzyloxy)phenyl)-2-bromo-3-((tert-butyldimethylsilyl)oxy)propanoate 25. The product can be further treated with lithium borohydride (LiBH₄) in THF to give the desired primary alcohol, (2R,3R)-3-(3,4-bis(benzyloxy)phenyl)-2-bromo-3-((tert-butyldimethylsilyl)oxy)propan-1-ol 26. Finally, the compound 26 can undergo an epoxidation reaction with sodium hydroxide (NaOH) to afford the anti-epoxide intermediate, (S)-(3,4-bis(benzyloxy)phenyl)((R)-oxiran-2-yl)methanol 19. Further steps can be the same as Steps 3-8 in Scheme XXXIV of Example 17.

### Example 19: Alternate Synthesis of Anti-Epoxide Intermediate

The compound tartaric acid can be used as a starting material to arrive at the desired anti-epoxide intermediate. The steps of this synthesis are shown in Scheme XXXVI and further described below.

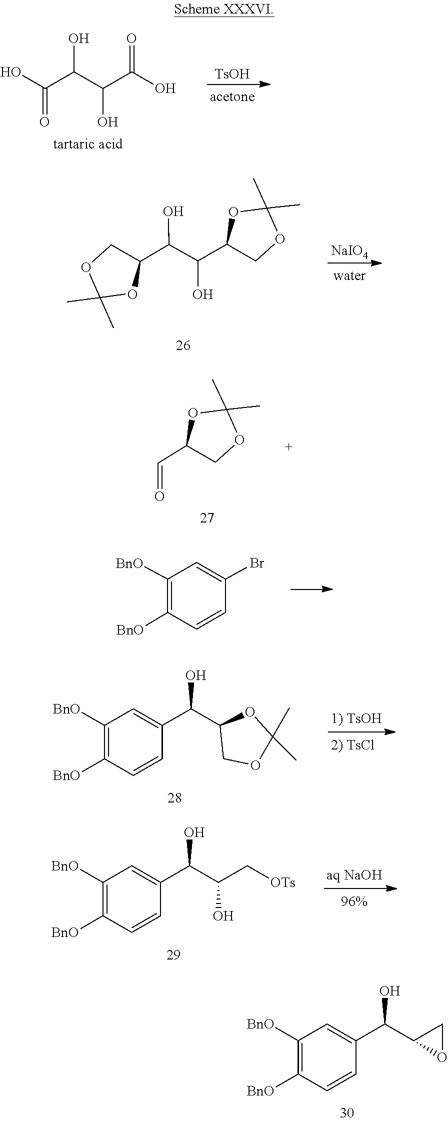

The compound tartaric acid can undergo a reaction with tosylic acid and acetone to give the desired dimer, 1,2-bis((S)-2,2-dimethyl-1,3-dioxolan-4-yl)ethane-1,2-diol 26. The dimer 23 can then undergo an oxidation with sodium periodate in water to afford the desired aldehyde, (S)-2,2-dimethyl-1,3-dioxolane-4-carbaldehyde 27. The aldehyde 27 can then undergo an addition reaction with (S)-2,2-dimethyl-1,3-dioxolane-4-carbaldehyde to give the desired secondary alcohol, (R)-(3,4-bis(benzyloxy)phenyl)((S)-2,2-dimethyl-1,3-dioxolan-4-yl)methanol 28. The secondary alcohol can then be subjected to tosylic acid followed by tosylic chloride to give the desired tosylated product, (2S,3R)-3-(3,4-bis(benzyloxy)phenyl)-2,3-dihydroxypropyl 4-methylbenzenesulfonate 29. Finally, the compound 29 can undergo an epoxidation reaction with sodium hydroxide (NaOH) to afford the anti-epoxide intermediate, (R)-(3,4-bis(benzyloxy)phenyl)((S)-oxiran-2-yl)methanol 30. Further steps can be the same as Steps 3-8 in Scheme XXXIV of Example 17.

Although the presently disclosed subject matter and its advantages have been described in detail, it should be understood that various changes, substitutions and alterations can be made herein without departing from the spirit and scope of the disclosed subject matter. Moreover, the scope of the present application is not intended to be limited to the particular embodiments of the process, and composition of matter, methods and processes described in the specification. As one of ordinary skill in the art will readily appreciate from the disclosed subject matter of the presently disclosed subject matter, processes, compositions of matter, methods, or steps, presently existing or later to be developed that perform substantially the same function or achieve substantially the same result as the corresponding embodiments described herein may be utilized according to the presently disclosed subject matter. Accordingly, the appended claims are intended to include within their scope such processes, machines, manufacture, compositions of matter, methods, or steps.

Various patents, patent applications, publications, product descriptions, protocols, and sequence accession numbers are cited throughout this application, the inventions of which are incorporated herein by reference in their entireties for all purposes.

What is claimed is:

1. A method for preparing (−)-epicatechin, the method comprising:
a) providing a compound of Formula V: 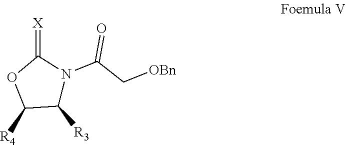
wherein X is O or S, R₃ is Ph, Bn, or CH(CH₃)₂, and R₄ is Ph or H, and further providing a compound of Formula II: 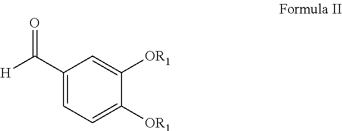
wherein R₁ is MOM or Bn;
b) coupling the compounds of Formula II and V to form a first intermediate, wherein the first intermediate comprises a secondary alcohol;
c) protecting the secondary alcohol in the first intermediate to form a second intermediate;
d) reducing the second intermediate to form a third intermediate, wherein the third intermediate comprises a primary alcohol;
e) removing the Bn group of Formula V to form a fourth intermediate;
f) selectively forming a sulfonate ester from the primary alcohol of the fourth intermediate to form a fifth intermediate;
g) epoxidizing the fifth intermediate to form a sixth intermediate;
h) alkylating the sixth intermediate to form a seventh intermediate, wherein the seventh intermediate comprises an alcohol;
i) protecting the alcohol of the seventh intermediate to form an eighth intermediate;
j) cyclizing the eighth intermediate to form a ninth intermediate;
k) deprotecting the ninth intermediate to form a tenth intermediate;
l) deprotecting the tenth intermediate to form (−)-epicatechin.

2. The method of claim 1, wherein X is S and R₃ is Ph.

3. The method of claim 1, wherein R₁ is MOM.

4. The method of claim 1, wherein the alcohol of the first intermediate is protected with a silyl group.

5. The method of claim 4, wherein the silyl group is t-butyldimethylsilyl.

6. The method of claim 1, wherein the reducing agent lithium borohydride is used for (d).

7. The method of claim 1, wherein a palladium catalyst in the presence of hydrogen gas is used for (e).

8. The method of claim 1, wherein the sulfonate ester of (f) is —OTs, —OMes, or —OTf.

9. The method of claim 8, wherein the sulfonate ester of (f) is —OTs.

10. The method of claim 1, wherein the sixth intermediate is alkylated with a compound of Formula VII: 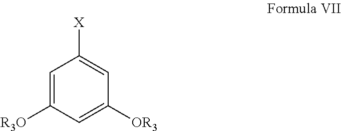
wherein X is F, Br, I, Cl, N, S, or B and R₃ is a Bn or MOM protecting group, to form the seventh intermediate.

11. The method of claim 10, wherein X is F and R₃ is Bn.

12. The method of claim 1, wherein the seventh intermediate is protected with a MOM or Bn protecting group.

13. The method of claim 12, wherein the seventh intermediate is protected with a MOM protecting group.

14. The method of claim 1, wherein the eighth intermediate is selectively deprotected before (j).

15. The method of claim 1, wherein the deprotection (k) or (1) is a debenzylation.

16. The method of claim 15, wherein the debenzylation uses a palladium catalyst in the presence of hydrogen gas.

17. The method of claim 1, wherein the sixth intermediate is the syn-epoxide intermediate.

18. A method of preparing (−)-epicatechin, the method comprising:
a) providing (S)-2-(benzyloxy)-1-(4-phenyl-2-thioxooxazolidin-3-yl)ethan-1-one and 3,4-bis(methoxymethoxy)benzaldehyde;
b) adding titanium tetrachloride to produce (2S,3R)-2-(benzyloxy)-3-(3,4-bis(methoxymethoxy)phenyl)-3-hydroxy-1-((S)-4-phenyl-2-thioxooxazolidin-3-yl)propan-1-one;
c) protecting (2S,3R)-2-(benzyloxy)-3-(3,4-bis(methoxymethoxy)phenyl)-3-hydroxy-1-((S)-4-phenyl-2-thioxooxazolidin-3-yl)propan-1-one with TBSCl to produce (2S,3R)-2-(benzyloxy)-3-(3,4-bis(methoxymethoxy)phenyl)-3-((tert-butyldimethylsilyl)oxy)-1-((S)-4-phenyl-2-thioxooxazolidin-3-yl)propan-1-one;
d) reducing (2S,3R)-2-(benzyloxy)-3-(3,4-bis(methoxymethoxy)phenyl)-3-((tert-butyldimethylsilyl)oxy)-1-((S)-4-phenyl-2-thioxooxazolidin-3-yl)propan-1-one with LiBH₄ to produce (2R,3R)-2-(benzyloxy)-3-(3,4-bis(methoxymethoxy)phenyl)-3-((tert-butyldimethylsilyl)oxy)propan-1-ol;
e) deprotecting (2R,3R)-2-(benzyloxy)-3-(3,4-bis(methoxymethoxy)phenyl)-3-((tert-butyldimethylsilyl)oxy)propan-1-ol with Pd/C under H₂ gas to produce (2R,3R)-3-(3,4-Bis(methoxymethoxy)phenyl)-3-((tert-butyldimethylsilyl)oxy)propane-1,2-diol;
f) tosylating (2R,3R)-3-(3,4-bis(methoxymethoxy)phenyl)-3-((tert-butyldimethylsilyl)oxy)propane-1,2-diol with tosylic acid to produce (2R,3R)-3-(3,4-bis(methoxymethoxy)phenyl)-3-((tert-butyldimethylsilyl)oxy)-2-hydroxypropyl 4-methylbenzenesulfonate;
g) epoxidizing (2R,3R)-3-(3,4-bis(methoxymethoxy)phenyl)-3-((tert-butyldimethylsilyl)oxy)-2-hydroxypropyl 4-methylbenzenesulfonate using potassium carbonate in methanol to produce ((R)-(3,4-Bis(methoxymethoxy)phenyl)((R)-oxiran-2-yl)methoxy)(tert-butyl)dimethylsilane;
h) alkylating ((R)-(3,4-bis(methoxymethoxy)phenyl)((R)-oxiran-2-yl)methoxy)(tert-butyl)dimethylsilane with (((5-fluoro-1,3-phenylene)bis(oxy))bis(methylene))dibenzene using n-butyl lithium to produce (1R,2R)-3-(2,4-Bis(benzyloxy)-6-fluorophenyl)-1-(3,4-bis(methoxymethoxy)phenyl)-1-((tert-butyldimethylsilyl)oxy)propan-2-ol;
i) protecting (1R,2R)-3-(2,4-bis(benzyloxy)-6-fluorophenyl)-1-(3,4-bis(methoxymethoxy)phenyl)-1-((tert-butyldimethylsilyl)oxy)propan-2-ol with MOMCl to produce (5R,6R)-5-{[2,4-bis(benzyloxy)-6-fluorophenyl]methyl}-6-[3,4-bis(methoxymethoxy)phenyl]-8,8,9,9-tetramethyl-2,4,7-trioxa-8-siladecane;
k) deprotecting (5R,6R)-5-{[2,4-bis(benzyloxy)-6-fluorophenyl]methyl}-6-[3,4-bis(methoxymethoxy)phenyl]-8,8,9,9-tetramethyl-2,4,7-trioxa-8-siladecane with TBAF to product (1R,2R)-3-(2,4-Bis(benzyloxy)-6-fluorophenyl)-1-(3,4-bis(methoxymethoxy)phenyl)-2-(methoxymethoxy)propan-1-ol;
l) cyclizing (1R,2R)-3-(2,4-bis(benzyloxy)-6-fluorophenyl)-1-(3,4-bis(methoxymethoxy)phenyl)-2-(methoxymethoxy)propan-1-ol using potassium hydride to product (2R,3R)-5,7-Bis(benzyloxy)-2-(3,4-bis(methoxymethoxy)phenyl)-3-(methoxymethoxy)chromane;
m) deprotecting (2R,3R)-5,7-bis(benzyloxy)-2-(3,4-bis(methoxymethoxy)phenyl)-3-(methoxymethoxy)chromane with HCl to produce 4-((2R,3R)-5,7-Bis(benzyloxy)-3-hydroxychroman-2-yl)benzene-1,2-diol; and
n) deprotecting 4-((2R,3R)-5,7-bis(benzyloxy)-3-hydroxychroman-2-yl)benzene-1,2-diol with Pd/C under H₂ to give (−)-epicatechin.
